(19) United States
(12) Patent Application Publication
(10) Pub. No.: US 2022/0073642 A1
(43) Pub. Date: Mar. 10, 2022

(54) METHODS OF USING SURUFATINIB IN TREATING ADVANCED PANCREATIC AND EXTRA-PANCREATIC NEUROENDOCRINE TUMORS

(71) Applicant: HUTCHISON MEDIPHARMA LIMITED, Shanghai (CN)

(72) Inventors: Wei-Guo SU, Shanghai (CN); Jing LI, Shanghai (CN)

(73) Assignee: HUTCHISON MEDIPHARMA LIMITED

(21) Appl. No.: 17/467,714

(22) Filed: Sep. 7, 2021

(30) Foreign Application Priority Data
Sep. 7, 2020 (CN) PCT/CN2020/113772

(51) Int. Cl. C07K 16/30; A61K 45/06; A61P 35/00

(52) U.S. Cl. CPC C07K 16/303; A61K 2039/505; A61P 35/00; A61K 45/06

(57) ABSTRACT

Disclosed herein are methods of using surufatinib in treating pancreatic and extra-pancreatic neuroendocrine tumors and advanced well-differentiated pancreatic and extra-pancreatic neuroendocrine tumors; the methods disclosed comprise administering to the patient an effective amount of surufatinib or a pharmaceutically acceptable salt thereof.

### CROSS-REFERENCE TO RELATED APPLICATIONS

This application claims the priority under 35 U.S.C. § 119(a) of International Application No. PCT/CN2020/113772, filed Sep. 7, 2020. The content of this application is incorporated herein by reference in its entirety.

### FIELD OF THE DISCLOSURE

The present disclosure relates to methods of using surufatinib in treating advanced well-differentiated pancreatic and extra-pancreatic neuroendocrine tumors.

### BACKGROUND OF THE DISCLOSURE

Neuroendocrine tumors (NETs) are rare neoplasms arising from diffusive neuroendocrine cells of various organs. In the past four decades, the age-adjusted incidence rate increased 6.4-fold in the United States, from 1.09 per 100,000 persons in 1973 to 6.98 per 100,000 persons in 2012. In China, a similar trend was identified by a hospital-based, nationwide, retrospective epidemiological study of gastroenteropancreatic neuroendocrine neoplasms.

Treatment options for advanced, low or intermediate grade NETs include somatostatin receptor-targeting therapeutics, peptide receptor radionuclide therapy, systemic chemotherapies, targeted agents including sunitinib in pancreatic NETs, and everolimus in pancreatic, gastrointestinal, and bronchopulmonary NETs, and local-regional treatments. Nearly half of all patients with NETs have distant metastasis at initial diagnosis. The median survival time for patients with well-differentiated to moderately differentiated distant stage NETs varies by tumor origins, ranging from 103 months for small intestinal origin, to 60 months for pancreatic origin, and 14 months for colonic origin. Although NETs are highly vascularized neoplasms, those originating from diverse organs respond to antiangiogenesis treatment differently.

NETs arising in the pancreas can be distinguished from those arising else-where in the gastrointestinal tract. Pancreatic neuroendocrine tumors (NETs) are rare tumors arising from embryological neuroendocrine cells of the pancreas, which have been increasing in incidence globally over the past decade (see, Dasari et al., JAMA Oncol., 3:1335-42 (2017)). The median overall survival varies according to numerous factors, including age, disease stage, pathological grade and treatment received (see, e.g., Halfdanarson et al., Ann Oncol, 19:1727-33 (2008); Sackstein et al., Semin Oncol, 45:24958 (2018)). NETs of lower grade (1 or 2), based on pathological grading of Ki-67 index and mitotic rate of tumor cells, are usually less aggressive than those of higher grade. In the metastatic setting, the median overall survival of grade 1/2 pancreatic NETs was approximately 5 years, based on estimates for tumors diagnosed between 2000-2012 (see Dasari et al).

Although pancreatic neuroendocrine tumors (PNETs) were originally thought to arise from pancreatic islet cells, recent work supports the notion that the PNETs arise from stem-like nonislet ductal progenitor cell, sustaining the transition in nomenclature from islet cell carcinoma to pancreatic neuroendocrine tumor.

The current recommended systemic treatments for unresectable, metastatic, well-differentiated, grade 1 or 2 pancreatic NETs include somatostatin analogs (SSAs) (e.g., octreotide or lanreotide), interferon, cytotoxic chemotherapy, targeted kinase inhibitors (sunitinib and everolimus), and peptide receptor radionuclide therapy for somatostatin receptor-positive NETs (see, e.g. Rinke et al., J Clin. Oncol., 27:4656-4663, (2009); Caplin et al., N Engi J Med., 371:224-233 (2014)).

A number of novel agents and combinations of agents are under investigation for the treatment of NETs. These include a CDK4/6 inhibitor (palbociclib), immunotherapies (pembrolizumab, ipilimumab plus nivolumab), and a combination of immunotherapy with antiangiogenic inhibitors (atezolizumab with bevacizumab). To date, limited antitumor activity has been observed with CDK4/6 inhibitor or immunotherapies as single-agent therapy in well-differentiated NETs (see, e.g., Strosberg et al., Clin Cancer Res., 26:2124-2130 (2020); Patel et al., Clin Cancer Res., 26:2290-2296 (2020)). However, encouraging efficacy has been reported from a phase 2 study of the combination of atezolizumab and bevacizumab in 20 patients with well-differentiated pancreatic NETs, with objective response rate of 20% (95% CI, 6%, 44%) and median progression-free survival of 19.6 months (95% CI, 10.6, not reached) (see, Halperin et al., J Clin Oncol., 38(S4): 619 (2020)).

The targeting of the angiogenesis mechanism is an established, effective treatment strategy for pancreatic NETs. Well-differentiated pancreatic NETs upregulate HIF-1α, which can lead to increased tumor vascularization. The activation of HIF-1α is driven by a genetic inactivation of the Von Hippel-Lindau protein and are associated with stimulation of hypoxic conditions that are typical in gastroenteropancreatic NETs (GEP-NET) cellular environments (see, Couvelard et al., Br J Cancer, 92:94-101 (2005)). The positive results of the phase 3 study of sunitinib, an agent targeting multiple kinases including vascular endothelial growth factor receptors (VEGFRs), in pancreatic NETs support the potential benefits of targeting tumor angiogenesis (see, Raymond et al., N Engl J Med, 364:5011-5013 (2011)). However, the primary and acquired resistance to sunitinib limits the clinical benefit for NETs patients (see, Pozas et al., Int J Mol Sci. 20:4949 (2019)). New drugs are needed in pancreatic NETs population.

Extra-pancreatic neuroendocrine neoplasms (extra-pancreatic NETs) originate in organs or tissue other than pancreas, of which gastrointestinal neuroendocrine tumors (GI-NETs), found in the stomach, duodenum, small intestines, appendix, cecum, colon and rectum, are the most common. Systemic treatment options for advanced well-differentiated (i.e. grade 1 or 2) extra-pancreatic NETs include somatostatin analogs (SSAs) (see, e.g. Rinke et al., J Clin. Oncol., 27:4656-4663, (2009)), chemotherapy, everolimus (for non-functional NETs of gastrointestinal or lung origin) (see, Yao et al., Lancet 387:968-977 (2016)), and peptide receptor radionuclide therapy (for somatostatin receptor-positive gastroenteropancreatic NETs) (see, Strosberg et al., N Engl. J Med., 376:125-135 (2017)). Sunitinib, a multi-targeted tyrosine kinase inhibitor that targets platelet-derived growth factor receptor, vascular endothelial growth factor receptors (VEGFRs) and other receptors, is currently approved for the treatment of advanced pancreatic NETs (see, e.g., Raymond et al., N Engl. J Med. 364:501-513 (2011)). Although several other drugs targeting the vascular endothelial growth factor (VEGF) pathway have shown antitumor activity in extra-pancreatic NETs (see, e.g., Yao et al., J Clin. Oncol. 35:1695-1703 (2017)), the efficacy of anti-angiogenic inhibitors are yet to be demonstrated in a well-controlled phase 3 study.

Anti-VEGF signaling pathway is a proven strategy for the treatment of pancreatic NETs. However, efficacy in extra-pancreatic NETs has not yet been proven. (See, Raymond E, et al. N Engl. J Med. 364:501-513 (2011)). Fibroblast growth factor (FGF) 2 was shown to be a potent mediator in antiangiogenesis resistance development, and inhibiting FGF receptor signaling could overcome resistance. (See, Tran et al. Mol. Cell Biol. 36:1836-1855 (2016)). Preclinical cancer models also showed that macrophages, usually recruited and activated by colony-stimulating factor 1 receptor (CSF-1R), played a proangiogenic role in the tumor microenvironment. Furthermore, eliminating tumor-associated macrophages by inhibiting CSF-1R led to decreased neoangiogenesis. (See, Ries et al. Cancer Cell 25:846-859 (2014)). Therefore, targeting multiple kinases to simultaneously block VEGFR-, FGFR-, and CSF-1R-mediated pathways may be a more effective method of preventing tumor angiogenesis and tumor immune evasion and therefore represents an attractive approach for cancer therapy.

Surufatinib (HMPL-012, formerly sulfatinib), namely N-(2-(dimethylamino)ethyl)-1-(3-((4-((2-methyl-1H-indol-5-yl)oxy)pyrimidin-2-yl)amino)phenyl)-methanesulfonamide, or a pharmaceutically acceptable salt thereof, having the formula (1)

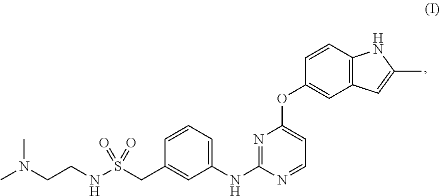

was disclosed in U.S. patent application Ser. No. 13/510,249 (the '249 application), which is a national stage of PCT/CN2010/078997, filed Nov. 23, 2010, now issued as U.S. Pat. No. 8,658,658 (the '658 patent). The '658 patent is incorporated herein by reference in its entirety.

Surufatinib is a potent, orally bioavailable small-molecule tyrosine kinase inhibitor (TKI), selectively targeting vascular endothelial growth factor receptors (VEGFR) 1, 2, and 3, fibroblast growth factor receptor type 1 (FGFR1), and colony stimulating factor-1 receptor (CSF-1R), with potential antineoplastic and anti-angiogenic activities.

Applicant has clinically proven the antitumor efficacy and manageable toxicities of surufatinib in treating patients with advanced NETs, including both pancreatic NETs and extra-pancreatic NETs. (See, e.g., Xu et al. Clin. Cancer Res. 25(12):3486-3494 (2019); PCT application No. PCT/CN2016/106404, which is incorporated by reference in its entirety).

There, however, remains unmet medical needs to develop novel therapeutic drugs in the treatment of advanced well-differentiated NETs, including both pancreatic and extra-pancreatic NETs. The present inventors surprisingly found that, by administering to the patient an effective amount of surufatinib or a pharmaceutically acceptable salt thereof, it is possible to improve PFS of patients with advanced pancreatic and extra-pancreatic NETs, and effectively treat advanced well-differentiated pancreatic and extra-pancreatic NETs.

### SUMMARY OF THE DISCLOSURE

In one aspect, the present disclosure provides a method of treating pancreatic neuroendocrine tumors or advanced well-differentiated extra-pancreatic neuroendocrine tumors in a patient in need thereof, the method comprising administering to the patient an effective amount of surufatinib or a pharmaceutically acceptable salt thereof.

In some embodiments, the patient was previously treated with no more than two prior first-line or second-line antitumor treatment chosen from somatostatin analogues (SSA), interferon, peptide receptor radionuclide therapy (PRRT), mammalian rapa Mycomycin target protein (mTOR) inhibitors and chemotherapy; and after the first-line or second-line antitumor treatment, the patient's neuroendocrine tumors continued to progress, wherein the patient with functioning NETs requiring treatment with long-acting SSAs, progression on prior VEGF/VEGFR inhibitors, or unstable or uncontrolled brain metastases were excluded.

In some embodiments, the patient does not have moderate or severe liver dysfunction. In some embodiments, the patient does not have moderate or severe renal insufficiency.

In some embodiments, the effective amount of surufatinib is a dose of 300 mg, administered orally to the patient once daily (QD). In some embodiments, the effective amount of surufatinib is a dose of less than 300 mg, administered orally to the patient QD.

In some embodiments, the effective amount of surufatinib is a dose of ranging from about 200 mg to about 300 mg. In some embodiments, the effective amount of surufatinib is a dose of about 200 mg. In some embodiments, the effective amount of surufatinib is a dose of about 250 mg. In some embodiments, the effective amount of surufatinib is a dose of about 200 mg, about 250 mg, about 300 mg, or a combination thereof.

In some embodiments, the effective amount of surufatinib is a dose taken as a single administration per day. In some embodiments, the administering step occurs at the same time every day.

In some embodiments, administrating surufatinib occurs continuously. In a further embodiment, administering surufatinib is continuous over a treatment cycle and the treatment cycle continues until the neuroendocrine tumor progression or the patient suffers from intolerable toxicity of surufatinib. In a further embodiment, the treatment cycle is up to about 4 weeks.

In some embodiments, the effective amount of surufatinib is a dose administered QD and further comprises adjusting the dose according to the safety and tolerability of the patient. In some further embodiments, adjusting the dose is chosen from dose interruption, dose reduction, dose discontinuation, and no dose adjustment.

In a further embodiment, adjusting the dose is dose interruption and dose interruption occurs when the patient meets one or more criteria chosen from: Grade 2 bleeding from any part, 24-hour urine protein quantity ≥2.0 g, Grade 2 acute renal injury, increased Grade 2 transaminase in combination with increased Grade 1 bilirubin, and adverse reactions of Grade 3 or Grade 4 except those requiring permanent discontinuation.

In another embodiment, adjusting the dose is dose reduction and dose reduction occurs when an adverse reaction resolves to ≤Grade 1 within 4 weeks, a first dose is adjusted to 250 mg of surufatinib QD and a second dose is adjusted to 200 mg of surufatinib QD; and when a dose of 200 mg of surufatinib QD is still intolerable, a dose adjustment to 200 mg surufatinib QD for 3 weeks on and 1 week off.

In another embodiment, adjusting the dose is dose discontinuation and dose discontinuation occurs when the patient meets one or more criteria chosen from: hemorrhage or gastrointestinal perforation ≥Grade 3; nephrotic syndrome or hypertension crisis; transaminase ≥3×ULN in combination with bilirubin increased to ≥2×ULN; increased Grade 4 transaminase in combination with increased Grade 4 bilirubin; and arterial thrombosis of any grade.

In another aspect, the present disclosure provides a method of treating advanced well-differentiated pancreatic neuroendocrine tumors in a patient in need thereof, the method comprising administering to the patient a pharmaceutical composition comprising surufatinib or a pharmaceutically acceptable salt thereof and at least one additional component chosen from pharmaceutically acceptable carriers, pharmaceutically acceptable vehicles, and pharmaceutically acceptable excipients

In an additional aspect, the present disclosure provides a method of treating extra-pancreatic neuroendocrine tumors or advanced well-differentiated extra-pancreatic neuroendocrine tumors in a patient in need thereof, the method comprising administering to the patient an effective amount of surufatinib or a pharmaceutically acceptable salt thereof.

In some embodiments, the patient was previously treated with one or more first-line or second-line antitumor treatment chosen from one or more of somatostatin analogues (SSA), interferon, peptide receptor radionuclide therapy (PRRT), mammalian rapa Mycomycin target protein (mTOR) inhibitors and chemotherapy; and after the first-line or second-line antitumor treatment, the patient's neuroendocrine tumors continued to progress.

In some embodiments, the patient does not have moderate or severe liver dysfunction. In some embodiments, the patient does not have moderate or severe renal insufficiency.

In some embodiments, the effective amount of surufatinib is a dose of 300 mg, administered orally to the patient once daily (QD). In some embodiments, the effective amount of surufatinib is a dose of less than 300 mg, administered orally to the patient QD.

In some embodiments, the effective amount of surufatinib is a dose of ranging from about 200 mg to about 300 mg. In some embodiments, the effective amount of surufatinib is a dose of about 200 mg. In some embodiments, the effective amount of surufatinib is a dose of about 250 mg. In some embodiments, the effective amount of surufatinib is a dose of about 200 mg, about 250 mg, about 300 mg, or a combination thereof.

In some embodiments, the effective amount of surufatinib is a dose taken as a single administration per day. In some embodiments, the administering step occurs at the same time every day.

In some embodiments, administrating surufatinib occurs continuously. In a further embodiment, administering surufatinib is continuous over a treatment cycle and the treatment cycle continues until the neuroendocrine tumor progression or the patient suffers from intolerable toxicity of surufatinib. In a further embodiment, the treatment cycle is up to about 4 weeks.

In some embodiments, the effective amount of surufatinib is a dose administered QD and further comprises adjusting the dose according to the safety and tolerability of the patient. In some further embodiments, adjusting the dose is chosen from dose interruption, dose reduction, dose discontinuation, and no dose adjustment.

In a further embodiment, adjusting the dose is dose interruption and dose interruption occurs when the patient meets one or more criteria chosen from: Grade 2 bleeding from any part, 24-hour urine protein quantity ≥2.0 g, Grade 2 acute renal injury, increased Grade 2 transaminase in combination with increased Grade 1 bilirubin, and any adverse reactions of Grade 3 or Grade 4 except those requiring permanent discontinuation. In a further embodiment, administration is reinitiated when one or more of the criteria resolves to ≤Grade 1 within one week after the dose interruption.

In another embodiment, adjusting the dose is dose reduction and dose reduction occurs when an adverse reaction resolves to ≤Grade 1 within 4 weeks, a first dose is adjusted to 250 mg of surufatinib QD and a second dose is adjusted to 200 mg of surufatinib QD; and when a dose of 200 mg of surufatinib QD is still intolerable, a dose adjustment to 200 mg surufatinib QD for 3 weeks on and 1 week off.

In another embodiment, adjusting the dose is dose discontinuation and dose discontinuation occurs when the patient meets one or more criteria chosen from: hemorrhage or gastrointestinal perforation a ≥Grade 3; nephrotic syndrome or hypertension crisis; transaminase ≥3×ULN in combination with bilirubin increased to ≥2×ULN; increased Grade 4 transaminase in combination with increased Grade 4 bilirubin; and arterial thrombosis of any grade.

In some embodiments, the method further comprises adjusting the effective amount of surufatinib administered per day according to a proteinuria level in the patient.

In a further embodiment, adjusting the dose is no dose adjustment and no dose adjustment occurs when the patient meets one or more criteria chosen from: when urinalysis shows protein+ and 24-hour urine protein quantity is less than 1.0 g, and when urinalysis shows protein 2+ or 3+ and 24-hour urine protein quantity is 1.0-2.0 g, excluding 2.0 g.

In another embodiment, adjusting the effective amount of surufatinib occurs when the patient meets one or more criteria chosen from: when a first 24-hour urine protein quantity ≥2.0 g occurs, a dose interruption applies, and the dose of surufatinib is reduced to 250 mg if the test results resolve to ≤Grade 1 within 4 weeks; when a second 24-hour urine protein quantity ≥2.0 g occurs, the dose interruption applies, and the dose of surufatinib is reduced to 200 mg if the test results resolve to ≤Grade 1 within 4 weeks; and when a third 24-hour urine protein quantity ≥2.0 g occurs, the dose interruption applies, and the dose of surufatinib is reduced to 200 mg with 3 weeks on and 1 week off if the test results resolve to ≤Grade 1 within 4 weeks, or dose discontinuation applies.

In another aspect, adjusting the effective amount of surufatinib follows the general guidance as below:

| Dose Modification | Adverse Reaction |
|---|---|
| Drug interruption | Grade 2 bleeding from any part |
| | 24-hour urine protein quantity ≥2.0 g |
| | Grade 2 acute renal injury |
| | Grade 2 transaminase increased combined |
| | with Grade 1 bilirubin increased |
| | Adverse reactions of Grade 3 or Grade 4 (except |
| | those requiring permanent discontinuation) |
| Dose reduction | The adverse reaction resolves to ≤Grade 1 or |
| | baseline level within 4 weeks after |
| | Surufatinib interruption |
| Drug | Hemorrhage or gastrointestinal |
| discontinuation | perforation ≥Grade 3 |
| | Nephrotic syndrome or hypertension crisis |
| | Transaminase ≥3 × ULN combined with |
| | bilirubin increased |
| | to ≥2 × ULN |
| | Grade 4 transaminase increased combined |
| | with Grade 4 bilirubin increased |
| | Arterial thrombosis of all grades |

The severity of adverse reactions was graded by the National Cancer Institute Common Terminology Criteria for Adverse Events version 4.03 (NCI CTCAE v4.03).

In another aspect, the present disclosure provides a method of treating advanced well-differentiated extra-pancreatic neuroendocrine tumors in a patient in need thereof, the method comprising administering to the patient a pharmaceutical composition comprising surufatinib or a pharmaceutically acceptable salt thereof and at least one additional component chosen from pharmaceutically acceptable carriers, pharmaceutically acceptable vehicles, and pharmaceutically acceptable excipients.

### BRIEF DESCRIPTION OF THE DRAWINGS

FIG. 1 illustrates the enrollment, randomization, and treatment as cut-off date of 11 Nov. 2019.

FIG. 2A shows Kaplan-Meier curves of progression-free survival as assessed by investigators.

FIG. 2B shows progression-free survival as assessed by Blinded Independent Image Review Committee (BIIRC).

FIG. 2C shows a forest plot of subgroup analyses based on investigators' assessment.

FIG. 3 illustrates the study design of Phase III (SANET-ep) clinical trial.

FIG. 4 illustrates the enrollment, randomization, and treatment as cut-off date of 31 Mar. 2019.

FIG. 5A shows Kaplan-Meier curves of progression-free survival as assessed by investigators.

FIG. 5B shows progression-free survival as assessed by Blinded Independent Image Review Committee (BIIRC).

FIG. 5C shows progression-free survival based on the post-hoc adjudicated BIIRC assessment.

FIG. 5D shows a forest plot of subgroup analyses based on investigators' assessment.

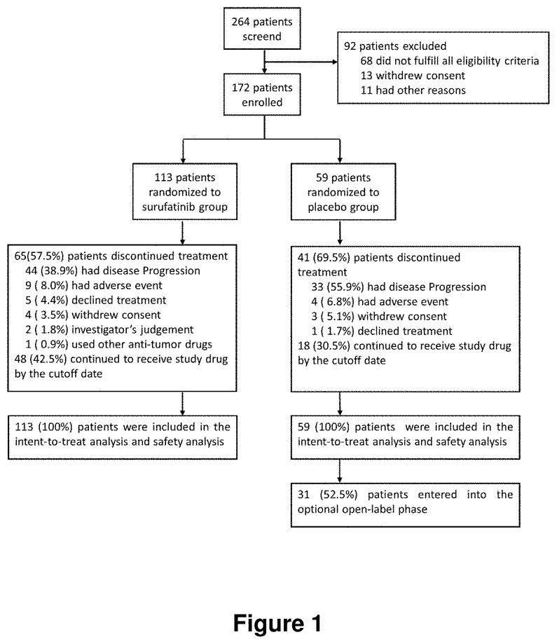

### DETAILED DESCRIPTION OF THE DISCLOSURE

The following terms, unless otherwise indicated, shall be understood to have the following meanings:

As used herein, including the claims, the singular forms of words, such as “a,” “an,” and “the,” include their corresponding plural references unless the context clearly dictates otherwise.

As used herein, the term “about” means approximately, in the region of, roughly, or around. When the term “about” is used in conjunction with a numerical range, it modifies that range or that numerical value by extending the boundaries above and below the numerical values set forth. In general, the term “about” is used herein to modify a numerical value above and below the stated value by a variance of ±20% or in some instances ±10%, or in some instances ±5%, or in some instances ±1%, or in some instances ±0.1% from the specified value, as such variations are appropriate to perform the disclosed methods.

The term “comprising” encompasses “including” as well as “consisting,” e.g. a composition “comprising” X may consist exclusively of X or may include something additional, e.g., X+Y. Throughout the specification and claims of the present disclosure, the terms “comprise,” “include,” “contain” and variations of them mean “including but not limited to”, and they are not intended to (and do not) exclude other moieties, additives, components, integers or steps.

As used herein, the term “treat,” “treating,” or “treatment”, when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art. of any disease or disorder refers in one embodiment, to ameliorating the disease or disorder (i.e., slowing or arresting or reducing the development of the disease or at least one of the clinical symptoms thereof). In yet another embodiment, “treat,” “treating,” or “treatment” refers to modulating the disease or disorder, either physically, (e.g., stabilization of a discernible symptom), physiologically, (e.g., stabilization of a physical parameter), or both.

As used herein, the term “inhibit,” “inhibition,” or “inhibiting” refers to the reduction or suppression of a given condition, symptom, or disorder, or disease, or a significant decrease in the baseline activity of a biological activity or process.

As used herein, the terms “administer,” “administering”, or “administration” as used herein refers to providing, giving, dosing and/or prescribing by either a health practitioner or authorized agent and/or putting into, taking or consuming by the patient or person himself or herself.

The term “patient” refers to a warm-blooded animal. In an embodiment, the patient is human. It may be a human who has been diagnosed and is in the need of treatment for a disease or disorder, as disclosed herein.

As used herein, a subject is “in need of” a treatment if such subject would benefit biologically, medically or in quality of life from such treatment.

An “effective amount,” “therapeutically effective amount,” or “pharmaceutically effective amount” are used interchangeably herein, and encompass an amount of a compound, formulation, material or composition, sufficient to treat or inhibit a symptom or sign of the medical condition. An effective amount for a particular patient may vary depending on factors, such as the condition being treated, the overall health of the patient, the method route and dose of administration and the severity of side effects. An effective amount can be the maximal dose or dosing protocol that avoids significant side effects or toxic effects. The effect will result in an improvement of a diagnostic measure or parameter by at least 5%, such as by at least 10%, further such as at least 20%, further such as at least 30%, further such as at least 40%, further such as at least 50%, further such as at least 60%, further such as at least 70%, further such as at least 80%, and even further such as at least 90%, wherein 100% is defined as the diagnostic parameter shown by a normal subject. An effective effective amount of surufatinib would be an amount that is, for example, sufficient to reduce a tumor volume, inhibit tumor growth, and improved progression-free survival of a patient.

The term “pharmaceutically acceptable” refers to those compounds, materials, compositions, and/or dosage forms which are suitable for use in contact with the tissues of human beings and animals without excessive toxicity, irritation, allergic response, or other problem or complication, commensurate with a reasonable benefit/risk ratio.

The term “pharmaceutically acceptable salts” can be formed, for example, as acid addition salts, preferably with organic or inorganic acids. Suitable inorganic acids are, for example, halogen acids, such as hydrochloric acid. Suitable organic acids are, e.g., carboxylic acids or sulfonic acids, such as fumaric acid or methanesulfonic acid. For isolation or purification purposes, it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. Any reference to the free compound herein is to be understood as referring also to the corresponding salt, as appropriate and expedient. The salts of the inhibitors, as described herein, are preferably pharmaceutically acceptable salts; suitable counter-ions forming pharmaceutically acceptable salts are known in the field.

The terms “tumor” or “cancer” refers to a disease characterized by the rapid and uncontrolled growth of aberrant cell proliferation. Cancer cells can spread locally or through the bloodstream and lymphatic system to other parts of the body. Examples of various cancers are, but are not limited to, leukemia, prostate cancer, renal cancer, liver cancer, brain cancer, lymphoma, ovarian cancer, lung cancer, cervical cancer, skin cancer, breast cancer, head and neck squamous cell carcinoma, pancreatic cancer, gastrointestinal cancer, colorectal cancer, triple-negative breast cancer, squamous cell cancer of the lung, squamous cell cancer of the esophagus, squamous cell cancer of the cervix, or melanoma. The terms “tumor” and “cancer” are used interchangeably herein, e.g., both terms encompass solid and liquid, e.g., diffuse or circulating tumors.

The terms “neuroendocrine tumor” (“NET”) as used herein refer to a cancer that arises from the neuroendocrine system, a diffuse system in which the nervous system and the hormones of the endocrine glands interact, or from non-endocrine cells by acquiring some of the properties of neuroendocrine cells through an oncogenic process such as Selective Tumor gene Expression of Peptides essential for Survival (STEPS) (see, North, Exper. Physiol. 85S:27S-40S (2000)). NET is an umbrella term for tumors that develop from cells in the endocrine and nervous systems, most commonly in the digestive and respiratory tracts. It can be split into two distinct populations based on molecular genetics and treatment options. Extra-pancreatic NET is the more common representing about 90% of NETs; the remainder are pancreatic NET (PNET).

The term of “first-line treatment” refers to the medication or treatment regimen that is used first, after diagnosis of a clinical condition. It is normally the treatment that has most data regarding its efficacy and safety for that specific condition. Most commonly, first-line treatment includes drugs which have been around for an extended period as there is plenty of experience with them, both in practice and through clinical studies.

“Second-line treatment” is a treatment for a disease or condition after the initial treatment (first-line treatment) has failed. Second-line or further lines of therapy (third-line, fourth-line, seventh-line, etc.) may be used for a few different reasons, for example, the first-line treatment doesn't work; the first-line treatment worked but has since stopped working; the first-line treatment has side effects that are not tolerated; or a new treatment becomes available that appears to be more effective than the present treatment.

The term “cycle” refers to a specific period of time expressed in days or months that is repeated on a regular schedule. The cycle as disclosed herein may be expressed in days. For example, the cycle can be, but is not limited to, 28 days, 30 days, 60 days, 90 days. Further for example, the “cycle” as referred to in the present disclosure is 28 days long. Such cycle can be repeated several time (e.g., 2 times, 3 times, 4 times, 5 times, etc.), each cycle may be the same or different length and can be repeated for a clinically meaningful result, i.e., the tumor growth is at least reduced, or controlled, or the tumor shrinks, and the adverse events are tolerable. In further embodiments, the cycle is about 28 days.

The term “dose” refers to measured portion of an active agent taken at any one time. There may be specific cases where higher or lower doses are appropriate; such doses do not depart from the context of the disclosure. According to the art, the dose appropriate for each patient is determined by, e.g., the physician according to the method of administration, the weight, the pathology, the body surface, the cardiac output and the response of said patient. Provided herein is a dose of surufatinib, i.e., the active agent, administered to the patient in need thereof. In some embodiments, the dose of surufatinib is about 300 mg administered orally to the patient once daily (QD). In some embodiments, the dose of surufatinib is less than 300 mg QD, for example, about 250 mg QD, about 200 mg QD, about 150 mg QD, about 100 mg QD, or a combination thereof. The dose of surufatinib may be adjusted according to the safety and tolerability of the patient, or be adjusted according to a proteinuria level in the patient.

As used here, the term “surufatinib” (formerly named sulfatinib”)refers to N-(2-(dimethylamino)ethyl)-1-(3-(4-(2-methyl-1H-indol-5-yloxy)pyrimidin-2-ylamino)phenyl)methanesulfonamide or N-(2-(dimethylamino)ethyl)-1-(3-(4-(2-methyl-1H-indol-5-yloxy)pyrimidin-2-ylamino)phenyl)methanesulfonamide. Surufatinib is a small-molecule kinase inhibitor which mainly acts on VEGFRs-1, 2, 3, the FGFR1 and CSF1R. Surufatinib is a study drug formulated as an odorless, white or off-white powder in capsules, with strengths of 50 mg and 200 mg. It is insoluble in water, and slightly soluble in methyl alcohol or ethyl alcohol. Herein, “surufatinib” may be referred to as “drug,” “active agent,” “a therapeutically active agent,” or “API.”

As used herein, the term “pharmaceutical composition” refers to an effective amount of surufatinib or of a pharmaceutically acceptable salt thereof, and also at least one pharmaceutically acceptable excipient and/or carrier. The pharmaceutical compositions may be given once daily, with or without food.

As used herein, the terms “pharmaceutically acceptable carrier,” “pharmaceutically acceptable vehicle,” “pharmaceutically acceptable excipient”, or “pharmaceutically acceptable excipient” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, fillers, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1289-1329 (1990)). Except insofar as any conventional carrier is incompatible with the active agent, its use in the therapeutic or pharmaceutical compositions is contemplated.

Suitable excipients for use in oral liquid dosage forms include, for example, dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance, tablets, pills or capsules may be coated with shellac, sugar or both. Additional excipients, for example, those sweetening, flavoring and coloring agents described above, may also be present.

The suitable unit administration forms comprise forms for oral administration, such as tablets, soft or hard gel capsules, powders, granules and oral solutions or suspensions, sublingual, buccal, intratracheal, intraocular or intranasal administration forms, forms for administration by inhalation, topical, transdermal, subcutaneous, intramuscular or intravenous administration forms, rectal administration forms, and implants.

In one embodiment of the invention, the dosage form of surufatinib is an oral solid dosage form. In one embodiment of the invention, the dosage form of surufatinib is a capsule or a tablet. In a further embodiment, the dosage form of surufatinib is a capsule. For example, dosage formulations are illustrated in International Publication No. WO 2016/188399A1, which is incorporated herein by reference in its entirety.

In some embodiments, the capsule of surufatinib is in a strength of 50 mg. When the dose of surufatinib is 300 mg, six capsules may be administered. When the dose of surufatinib is 250 mg, five capsules may be administered. When the dose of surufatinib is 200 mg, four capsules may be administered. When the dose of surufatinib is 150 mg, three capsules may be administered. When the dose of surufatinib is 100 mg, two capsules may be administered.

Previous Clinical Studies

Two clinical trials examining the effects of Surufatinib have been completed: (1) A dose-escalation phase I study of single agent Surufatinib in patients with advanced malignant solid tumors (NCT02133157); and (2) A phase Ib/II study of Surufatinib in patients with advanced neuroendocrine tumors (NETs) (NCT02267967).

A Dose-Escalation Phase I Study of Surufatinib (NCT02133157)

This multi-center, open-label, dose-escalation phase I study investigated the safety, pharmacokinetic characteristics, and preliminary anti-tumor activity of surufatinib in patients with advanced solid tumors. The primary objective was to determine the maximum tolerated dose (MTD), recommended phase II trial dose (RPTD), and the safety profile of Surufatinib when administered to patients with advanced malignant solid tumors. A secondary objective was to evaluate the pharmacokinetic profile of Surufatinib, and exploratory objectives included evaluating the ORR among patients given Surufatinib therapy and determining whether biomarkers for angiogenesis might reflect any clinical efficacy shown by Surufatinib. A modified Fibonacci 3+3 construct was used to design this dose escalation study and determine the MTD of Surufatinib. The results from this phase I study was published in Oncotarget (Jianming Xu, et al. Oncotarget. 8(26):42076-42086) and showed that surufatinib exhibited an acceptable safety profile and encouraging antitumor activity in patients with advanced solid tumors, particularly neuroendocrine tumors.

Phase Ib/II Study of Surufatinib in Treating Advanced Neuroendocrine Tumors (NCT02267967)

This multi-center, single-arm, open-label, phase Ib/II study investigated the efficacy, safety, tolerability, and pharmacokinetics of Surufatinib in treating advanced neuroendocrine tumors. The primary objective was to evaluate the efficacy and safety of surufatinib. Pharmacokinetics were evaluated as the secondary objective. The results from this phase Ib/II study was published in Clin Cancer Res. (Jianming Xu, et al. Clin Cancer Res. 25(12):3486-3494) and showed that surufatinib demonstrated a manageable and expected toxicity profile and has potential as a pharmacologic treatment for patients with pancreatic or extra-pancreatic neuroendocrine tumors, including those who have previously failed VEGFR inhibitors.

In conclusion, when used in clinical practice, any adverse events caused by Surufatinib are tolerable and can be easily managed.

### EXAMPLES

Neuroendocrine tumor cells produce and secrete large amounts of angiogenic factors (e.g., VEGF-A and VEGF-C) to promote neovascularzation in the tumor microenvironment. This activity plays a pivotal role in tumor proliferation and metastasis. Sunitinib has been shown to significantly prolong PFS times in patients being treated for NETs, suggesting that NETs are highly sensitive to anti-angiogenesis therapy.

However, antiangiogenesis drugs (e.g., sunitinib, bevacizumab and pazopanib) in the extra-pancreatic NET population have not shown adequate efficacy. Currently there are very limited options for the treatment of advanced extra-pancreatic NETs. Only SSAs (e.g., octreotide and lancreotide) and mTOR inhibitor (everolimus) have been approved by the FDA. While low anti-tumor proliferation activity can be achieved with SSA drugs, there is insufficient data to support the use of SSA drugs in progressive NET patients. Clinically, there are no effective drugs for the treatment of extra-pancreatic progressive NETs. Thus, the clinical demand for treatment of extra-pancreatic NETs is huge. Worldwide more clinically effective drugs are needed for patients with NETs to prolong survival.

Previous studies have proven that Surufatinib strongly inhibits tumor angiogenesis, effectively inhibiting activity of multiple protein kinases in tumor cells, and therefore inhibiting growth, local invasion and distant metastasis of tumor cells. Through preliminary clinical trial data, observations have been made that Surufatinib can be safely tolerated and shows good efficacy in some patients, especially those with NETs. At the extension stage of the Phase I dose escalation clinical study, 21 efficacy evaluable patients with NETs received Surufatinib, of which 8 patients achieved PR, 10 achieved SD, and 3 were not evaluable for response, with and ORR of 38.1% and DCR of 85.7%. In addition, similar efficacy trends have also been shown in the Phase Ib/II study of patients with NETs (Jianming Xu et al. Clin Cancer Res. 25(12):3486-3494). Furthermore, Surufatinib showed no significant treatment selectivity across the different NETs, showing extensive efficacy. Therefore, further clinical investigation of Surufatinib is valuable. Below are two further clinical trials with results demonstrating that Surufatinib prolonged progression-free survival and was well-tolerated in most patients with progressive, advanced pancreatic and extra-pancreatic NETs.

### Example 1: SANET-p Trial

1. Summary of Phase III Clinical Study (SANET-p Trial)

SANET-p (NCT02589821) is a randomized, double-blind, placebo-controlled Phase III study evaluating the efficacy and safety of surufatinib compared to placebo in patients with progressive, advanced (unresectable local progression or distant metastasis), well-differentiated pancreatic neuroendocrine tumors.

2. Study Design and Dose Administration

This study is a randomized, double-blind, placebo-controlled Phase III study evaluating the efficacy and safety of surufatinib in treating advanced, well-differentiated pancreatic neuroendocrine tumors.

The targeted population are progressive, locally advanced or distant metastatic, low- or intermediate grade (G1 or G2) pancreatic NETs patients. G1 is defined as <2 mitoses/10 high-power field [HPF] and/or <3% Ki-67 index; G2 is defined as 2-20/10 HPF and/or 3-20% Ki-67 index. If the mitotic ratio and Ki-67 index correspond to different grade, the higher grade should be used to assign classification. All patients must provide tumor samples for central pathology review to check as of whether the diagnostics and grade score comply with inclusion criteria. Patients must have radiological documentation of progression of disease within 12 months prior to randomization. Patients must have previously progressed no more than two types of systemic anti-tumor therapy, including long-acting somatostatin analogs (SSAs), interferon, PRRT (peptide receptor radionuclide therapy), mammalian target of rapamycin (mTOR) inhibitors or chemotherapy (all chemotherapies are deemed as a single therapy regardless of drug or time); patients who are unable or unwilling to receive such treatments are also eligible if all other criteria are met.

Double-Blinded Stage

Approximately 195 patients will be randomly assigned (in 2:1 ratio) to the following treatment groups based on interactive web response system IWRS:

Surufatinib, 300 mg, once daily (QD), orally

Placebo, 300 mg, once daily (QD), orally

Continuous administration (every 4 weeks as a treatment cycle). Surufatinib can be taken with or without food and should be taken as a single administration per day. It is recommended to take the drug at the same time every day. If the patient vomits after dosing surufatinib, there is no need to make up. If a dosage is missed, do not make up the missed dose in the next day, and take the next dose as scheduled.

During the treatment, physician should closely monitor the patients and adjust dosage according to the safety and tolerability of individual patients, including dose interruption, dose reduction or permanently discontinue treatment. The dose adjustment should follow the guidance of interruption first and then dose reduction.

After dose interruption, if the adverse reaction resolves to ≤Grade 1 within 4 weeks, it is recommended to adjust the dose under the instruction of the physician:

the first dose is adjusted to 250 mg QD (5 capsules, each capsule containing 50 mg of surufatinib);

the second dose is adjusted to 200 mg QD (4 capsules, each capsule containing 50 mg of surufatinib); if the dose of 200 mg QD is still intolerable, then the dose of 200 mg QD for 3 weeks on and 1 week off or permanently discontinue treatment should be considered.

The general guidance of dose modification is shown in Table 1 and Table 2.

TABLE 1
Dose Adjustment for Hematological Toxicity

| NCI CTCAE v4.03 Toxicity Grading | Action |
|---|---|
| Grade 1 or 2 | None |
| Grade 3 or 4^{b} | |
| Manageable/reversible after reduction | Withhold^{a} |
| Grade 3 or 4 decrease | Reduce to a lower dose level |
| to ≤Grade 1 or baseline | |
| level within 28 days | |
| Reoccurrence of Grade 3 toxicity | Reduce 1 dose level or |
| | discontinue treatment |
| Reoccurrence of Grade 4 toxicity | Discontinue treatment |
| Grade 3 or 4 | |
| Unmanageable/irreversible | Discontinue treatment |
| after reduction | |

^{a}Treatment is to be withheld until toxicity returns to ≤grade 1 or baseline level. Patients whose toxicity has been reduced for over 28 days can generally be discontinued from the study, unless the investigator determines that the patients can still benefit clinically from the study. In this case, the investigator is to discuss with the sponsor as to whether to keep the patient in the study or not.
^{b}If grade 3 or grade 4 leukocyte or neutrophils decrease and recover to ≤grade 1 or to baseline level within 28 days, administer the original dose. Only complicated neutropenia, including recurrent or persistent grade 4 neutropenia occurring for >7 days or concurrent with a fever or infection will require dose reduction.

TABLE 2
Dose Adjustment for Non-Hematological Toxicity

| NCI CTCAE v4.03 Toxicity Grading | Action |
|---|---|
| Grade 1 or 2 (Tolerable) | None |
| Grade 2 (intolerable) ^{d} | Withhold ^{c} |
| Toxicity decrease | Maintain previous dose |
| to ≤Grade 1 or baseline | |
| level within 28 days | |
| Recurrent | Reduce 1 dose level |
| Grade 3 or 4 ^{d} | |
| Manageable/reversible after reduction | Withhold ^{c} |
| Grade 3 toxicity decrease | Reduce 1 dose level |
| to ≤Grade 1 within 28 days | |
| Reoccurrence of Grade 3 toxicity | Reduce 1 dose level or |
| | discontinue treatment |
| Reoccurrence of Grade 4 toxicity | Discontinue treatment |
| Grade 3 or 4 | |
| Unmanageable/irreversible | Discontinue treatment |
| after reduction | |

^{c} Patients whose toxicity has been persistent without recovery for over 28 days can generally be discontinued from the study, unless the investigator determines that the patients can still benefit clinically from the study. In this case, the investigator is to discuss with the sponsor as to whether to keep the patient in the study.
^{d} For nausea, vomiting or diarrhea, supportive treatment is to be given first. If after the supportive treatment, the investigational product is still not tolerated, study drug is to be withheld until the toxicity decreases to ≤grade 1 or baseline level. For toxic events that the investigator considers unlikely to be serious or life threatening and that can be tolerated by patients, such as hyperuricemia, hypophosphatemia and alopecia laboratory results, the investigational treatment may stay at the same dose level without dose reduction, provided supportive care has been given to treat the event.

According to the randomized result, the patients will receive continuous oral treatment, every 4-week treatment cycle until progression of disease occurs, intolerable toxicity or other protocol-specified end-of-treatment criteria is met.

Other antitumor therapies are forbidden during the treatment. However, when patients have obvious NET functional symptoms and need to use somatostatin analogues (SSAs) to control symptoms, they can receive short-term SSAs treatment.

In accordance with RECIST 1.1, the tumor should be assessed every 8 weeks (±3 days) within the first year and every 12 weeks (±3 days) after the patient has been treated for more than one year.

Inclusion Criteria

The enrolled participants should meet all following criteria:

1. Adequately understand the study and voluntarily sign the Informed Consent Form;

2. Be at least 18 years old;

3. Based on central pathology review results, patients have a confirmed histologically pathology diagnosis of low- or intermediate grade (G1 or G2) advanced (unresectable or distant metastatic) PNET. G1 is defined as <2 mitoses/10 high-power field [HPF] and/or <3% Ki-67 index; G2 is defined as 2-20/10 HPF and/or 3-20% Ki-67 index. If the mitotic ratio and Ki-67 index correspond to different grade, the higher grade should be used to assign classification.

4. Have previously progressed on no more than two types of systemic anti-tumor therapy, including long-acting somatostatin analogs (SSAs), interferon, PRRT (peptide receptor radionuclide therapy), mTOR inhibitors or chemotherapy (chemotherapies were considered as one kind of regimen, regardless of medications and cycles); patients who are unable or unwilling to receive such treatments are also eligible;

5. Patients must have radiological documentation of progression of disease within 12 months prior to randomization.

6. Have measurable lesions (according to RECIST 1.1);

7. Absolute neutrophil count (ANC) of 21.5×10⁹/L, platelet count of ≥100×10⁹/L, and hemoglobin 29 g/dL;

8. Serum total bilirubin <1.5 times the upper limit of normal (ULN);

9. Patients who do not have liver metastasis, with alanine aminotransferase (ALT) and aspartate aminotransferase (AST) levels ≤2.5 times the ULN; and who do have liver metastatic, with ALT and AST ≤5 times ULN.

10. Serum creatinine <1.5 times ULN and creatinine clearance 260 ml/min;

11. International Normalized Ratio (INR) ≤1.5 ULN and activated partial thromboplastin time (APTT) ≤1.5 ULN.

12. Have a performance status (PS) of 0 or 1 on the Eastern Cooperative Oncology Group (ECOG) scale;

13. Have expected survival of more than 12 weeks;

14. Male or female patients with reproductive potential must agree to use an effective contraceptive method, for example, double-barrier device, condom, oral or injected birth control medication or intrauterine device, during the study and within 90 days after study treatment discontinuation. All female patients are considered to be fertile, unless the patient had natural menopause or artificial menopause or sterilization (such as hysterectomy, bilateral oophorectomy or ovarian irradiation).

Exclusion Criteria

Enrolled Participants Will be Excluded for any One of the Below Criteria:

1. High grade (G3) neuroendocrine cancer, adenocarcinoid, pancreatic islet cell carcinoma, goblet cell carcinoid, large cell neuroendocrine carcinoma and small cell carcinoma;

2. Functional NETs which need to be treated with long acting SSAs to control disease related syndromes, such as insulinoma, gastrinoma, glucagonoma, somatostatinoma, ACTHoma, VIPoma, accompanied by carcinoid syndrome, Zollinger-Ellison syndrome or other active symptoms;

3. Have received anti-VEGF/VEGFR targeted drugs and progressed upon these drugs;

4. Urinalysis shows urine protein a ≥2+ or 24-hour protein quantity test shows urinary protein ≥1 g;

5. Serum potassium, calcium (albumin-bound ionic or corrected) or magnesium exceeds the normal range with clinical significance;

6. Under anti-hypertension treatment, still uncontrolled hypertension, defined as: systolic blood pressure >140 mmHg or diastolic blood pressure >90 mmHg;

7. Gastrointestinal disease or condition that investigators suspect may affect drug absorption, including, but not limited to, active gastric and duodenal ulcers, ulcerative colitis and other digestive disease, gastrointestinal tumor with active bleeding, or other gastrointestinal conditions that may cause bleeding or perforation by investigator's discretion;

8. History or presence of a serious hemorrhage (>30 ml within 3 months), hemoptysis (>5 ml blood within 4 weeks) or a thromboembolic event (including transient ischemic attack) within 12 months;

9. Clinically significant cardiovascular disease, including but not limited to, acute myocardial infarction within 6 months prior to enrollment, severe/unstable angina pectoris or coronary artery bypass grafting, congestive heart failure according to the New York Heart Association (NYHA) classification ≥2; ventricular arrhythmias which needs drug treatment; LVEF (LVEF)<50%;

10. Mean corrected QT interval (QTc) ≥480 msec;

11. Other malignancies diagnosed within the previous 5 years, except basal cell carcinoma or cervical carcinoma in situ after radical resection;

12. Anti-tumor therapy received within 4 weeks prior to the initiation of the investigational treatment, including, but not limited to, chemotherapy, radical radiotherapy, targeted therapy, immunotherapy and anti-tumor Chinese medicine treatment, hepatic chemoembolization, cryoablation and radiofrequency ablation;

13. Palliative radiotherapy for a bone metastasis lesion within 2 weeks prior to the initiation of the investigational treatment;

14. Drugs containing St John's wort taken within 3 weeks prior to the first study treatment, or other strong inducers with CYP3A4 or strong inhibitors taken within two weeks prior to the first study treatment (see appendix 3);

15. Any clinically significant active infection, including, but not limited to, human immunodeficiency virus (HIV) infection;

16. History of clinically significant hepatic disease, including, but not limited to, known hepatitis B virus (HBV) infection with HBV DNA positive (copies ≥1 ×10⁴/ml); known Hepatitis C virus infection with HCV RNA positive (copies ≥1 ×10³/m); or liver cirrhosis, etc.

17. Surgery (except biopsy) within 28 days prior to the initiation of investigational treatment or unhealed surgical incision;

18. Brain metastases and/or spinal cord compression not treated by surgery and/or radiotherapy, and with no clinical imaging evidence of disease stability;

19. Toxicity from a previous anti-tumor treatment that does not return to Grade 0 or 1 (except for hair loss);

20. Received investigational treatments in other clinical studies within 4 weeks prior to enrollment:

21. Women who are pregnant or lactating;

22. Other disease, metabolic disorder, physical examination anomaly, abnormal laboratory result, or any other conditions are inappropriate for the use of the investigational product or affect interpretation of study results.

Endpoints

Therapeutic Endpoints:

1) Primary therapeutic endpoint: PFS (according to RECIST 1.1 criteria)

2) Secondary therapeutic endpoint: Objective Response Rate of the tumor (ORR), Disease Control Rate (DCR), Duration of Response (DoR), Time to Response (TTR) and Overall Survival (OS) (according to RECIST 1.1 criteria)

Exploratory Endpoints:

For patients whose serum CgA are abnormally elevated from two arms, the serum CgA and 24 h urine 5-HIAA level change of patients after treatment was compared with baseline.

The change of VEGF, FGF23 and other target-related biomarkers after treatment in plasma and/or tumor tissues compared with baseline

To determine the correlation between steady-state pharmacokinetics in vivo in patients (blood drug concentration based on C1D15 data, including but not limited to Cₘₐₓ, Cₘᵢₙ and AUCₛₛ) and tumor response, serum CgA level as well as AE incidence.

The score change of life quality questionnaire EORTC QLO-C30 and QLQ-GI.NET 21 from two arms after treatment were compared with baseline.

Results and Analysis

Patients and Treatments

As of the cut-off date (Nov. 11, 2019), a total of 172 patients were randomly enrolled in a 2:1 ratio into the study cohorts (113 patients in surufatinib cohort, 59 patients in placebo cohort).

The main baseline characteristics of the patients are shown in Table 3. The demographic data and baseline characteristics of the placebo cohort were generally consistent with those of the Surufatinib cohort.

TABLE 3
Demographic and Baseline Clinical Characteristics (Intent-to-treatment Set)

| | Surufatimb | | Placebo | |
|---|---|---|---|---|
| Characteristic | (N = 113) | | (N = 59) | |
| Age in years, median (range) | 51.0 | (25.0-75.0) | 48.0 | (20.0-77.0) |
| Male, n (%) | 60 | (53.1) | 28 | (47.5) |
| ECOG performance status, n (%) | | | | |
| 0 | 73 | (64.6) | 43 | (72.9) |
| 1 | 40 | (35.4) | 16 | (27.1) |
| Pathologic grade, n (%) | | | | |
| Grade 1 | 14 | (12.4) | 9 | (15.3) |
| Grade 2 | 99 | (87.6) | 50 | (84.7) |
| Function status, n (%) | | | | |
| Functioning | 11 | (9.7) | 3 | (5.1) |
| Non-functioning | 102 | (90.3) | 55 | (93.2) |
| Unknown | 0 | | 1 | (1.7) |
| Metastatic sites, n (%) | | | | |
| Liver | 108 | (95.6) | 54 | (91.5) |
| Lymph nodes | 42 | (37.2) | 21 | (35.6) |
| Lung | 9 | (8.0) | 3 | (5.1) |
| Bone | 16 | (14.2) | 3 | (5.1) |
| Other | 24 | (21.2) | 8 | (13.6) |
| Number of organs involved, n (%) | | | | |
| ≤2 | 51 | (45.1) | 34 | (57.6) |
| ≥3 | 62 | (54.9) | 25 | (42.4) |
| Time from diagnosis to randomization | | | | |
| ≤24 months | 67 | (59.3) | 36 | (61.0) |
| >24 months | 46 | (40.7) | 23 | (39.0) |
| Time from disease progression to randomization, n (%) | | | | |
| ≤3 months | 102 | (90.3) | 54 | (91.5) |
| >3 to ≤6 months | 9 | (8.0) | 4 | (6.8) |
| >6 to ≤12 months | 2 | (1.8) | 1 | (1.7) |
| Prior systemic antitumor drug for advanced disease, n (%) | | | | |
| Any prior systemic antitumor treatment | 74 | (65.5) | 39 | (66.1) |
| Prior somatostatin analog treatment | 48 | (42.5) | 28 | (47.5) |
| Prior systemic chemotherapy | 33 | (29.2) | 12 | (20.3) |
| Prior everolimus treatment | 12 | (10.6) | 4 | (6.8) |

The intent-to-treatment population which included all patients randomized.
ECOG = European Co-operative Oncology Group. SSA = somatostatin analog.

The median treatment duration was 229 days (range 3 to 1174) for the surufatinib group and 123 days (range from 5 to 1127) for placebo. The mean relative dose intensity (the ratio of actual to planned dose intensity) was 89.0% (standard deviation 13.5%) and 97.6% (standard deviation 5.3%) in the surufatinib and placebo groups, respectively.

At the time of data cut-off for interim analysis, 65 patients (57.5%) in the surufatinib group and 41 (69.5%) in the placebo group had discontinued double-blinded study treatment. The primary reasons for treatment discontinuation were disease progression (44 [38.9%] in surufatinib group versus 33 [55.9%] in placebo group).

Efficacy

At the time of data cutoff, 95 progression-free survival events had occurred, as assessed by investigators. The median progression-free survival follow-up time was 19.3 months (95% CI, 9.3, 19.4) in the surufatinib group and 11.1 months (95% CI, 5.7, 35.9) in the placebo group. The median investigator-assessed progression-free survival was 10.9 months (95% confidence interval [CI], 7.5, 13.8) in the surufatinib group versus 3.7 months (95% CI, 2.8, 5.6) in the placebo group (HR, 0.49; 95% CI, 0.32, 0.76; p=0.0011).

Progression free survival (PFS) as assessed by the investigator was significantly prolonged in Surufatinib cohort, the median PFS of which was 7.2 months longer than that in the placebo cohort, and the risk of disease progression/death was reduced by 50.9%. The result of BIIRC assessment was consistent with investigator's assessment. Objective response rate (ORR) of the secondary endpoint in the Surufatinib cohort was 19.2%, which was significantly superior to those in the placebo cohort (1.9%). The efficacy results are shown in Table 4.

TABLE 4
Efficacy summary (ITT set) (SANET-p) study

| | Surufatinib cohort | | Placebo cohort | |
|---|---|---|---|---|
| Efficacy endpoint | N = 113 | | N = 59 | |
| PFS (Investigator's | | | | |
| assessment) | | | | |
| Median (month) (95% CI) | 10.9 | (7.5, 13.8) | 3.7 | (2.8, 5.6) |
| HR (95% CI) | 0.491 (0.319, 0.755) | | | |
| p value¹ | 0.0011 | | | |
| PFS (BIIRC assessment) | | | | |
| Median (month) (95% CI) | 13.9 | (11.0, 24.9) | 4.6 | (3.6, 7.4) |

| | |
|---|---|
| HR (95% CI) | 0.339 (0.209, 0.549) |
| p value¹ | <0.0001 |

| | |
|---|---|
| N = 104 | N = 53 |

| ORR | | | | |
|---|---|---|---|---|
| ORR (%) (95% CI) | 19.2 | (12.2, 28.1) | 1.9 | (0.0, 10.1) |
| OR (95% CI) | 12.4 (1.8, 522.8) | | | |
| p value² | 0.0021 | | | |
| DCR | | | | |
| DCR (%) (95% CI) | 80.8 | (71.9, 87.7) | 66.0 | (51.7, 78.5) |

| | |
|---|---|
| OR (95% CI) | 2.1 (0.9, 4.8) |
| p value³ | 0.0774 |

| | |
|---|---|
| N = 15 | N = 1 |

| | | | |
|---|---|---|---|
| DoR | | | |
| Median (month) (95% CI) | 15.2 | (5.6, —) | 2.8 |
| TTR | | | |
| Median (month) (95% CI) | 3.9 | (1.9, 5.6) | 33.1 |

Abbreviations: CI = confidence interval, DCR-disease control rate, DoR = duration of response, HR = hazard ratio (Surufatinib/Placebo), ITT Set = intention to treat set, OR = odds ratio, ORR = objective response rate, PFS = Progression-free Survival, TTR = time to response.
¹Stratified Log-Rank test.
²Exact test.
³Stratified Cochran-Mantel-Haenszel test.

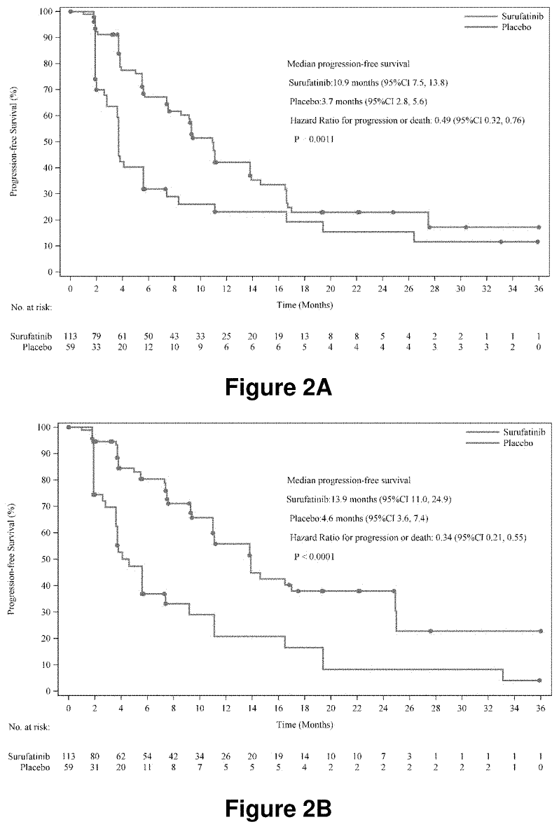

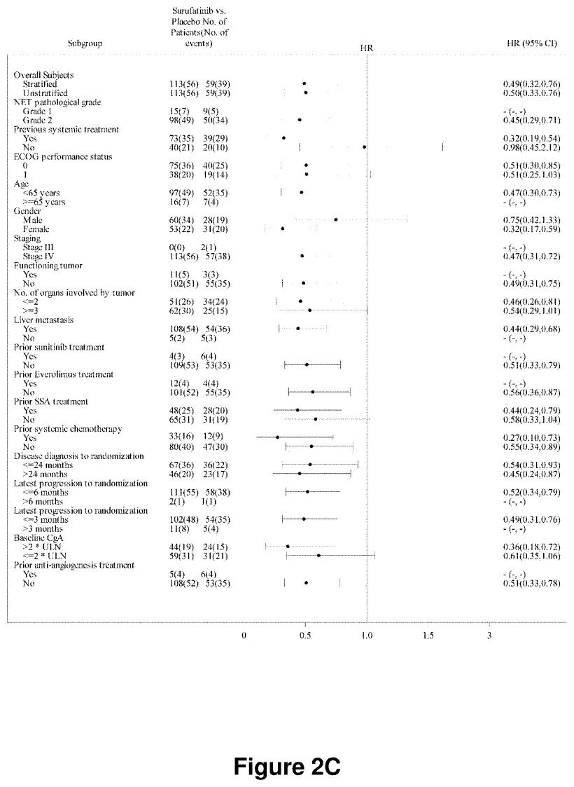

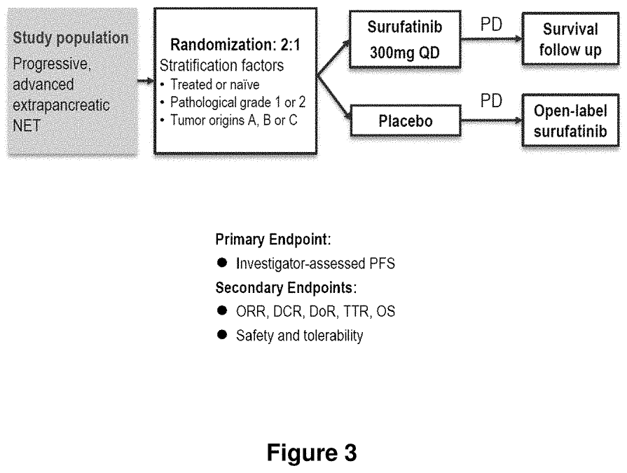

Surufatinib reduced the risk of disease progression or death by 51% compared with placebo (see FIG. 2A). Results of sensitivity analysis, BIIRC-assessed progression-free survival, were consistent with those of the primary outcome measure of investigator-assessed progression-free survival; median BIIRC-assessed progression-free survival was 13.9 months (95% CI, 11.0, 24.9) with surufatinib versus 4.6 months (95% CI, 3.6, 7.4) with placebo (HR, 0.34; 95% CI, 0.21, 0.55; p<0.0001) (see FIG. 2B). The progression-free survival benefit favored surufatinib across all major subgroups (see FIG. 2C). The study was terminated by the IDMC recommendation based on the positive interim analysis results.

FIG. 2A shows Kaplan-Meier curves of progression-free survival as assessed by investigators. Events of progression or death were observed in 56 patients (49.6%) in the surufatinib group and 39 (66.1%) in the placebo group. FIG. 2B shows progression-free survival as assessed by BIIRC. Events of progression or death were observed in 40 patients (35.4%) in the surufatinib group and 36 (61.0%) in the placebo group. A forest plot of subgroup analyses based on investigator assessment is shown in FIG. 2C.

In the interim intent-to-treat set, 20 patients in the surufatinib group and one patient in the placebo group achieved partial response, as assessed by the investigators, resulting in objective response rates of 19.2% (95% CI, 12.2, 28.1) versus 1.9% (95% CI, 0.0, 10.1) (p=0.0021), respectively. The median time to response and median duration of response are shown in Table 5. Trends in overall response by BIIRC assessment were similar to those reported by the investigators: objective response rate of 14.4% (95% CI, 8.3, 22.7) versus 1.9% (95% CI 0.0, 10.1) (p=0.0123), respectively (Table 4).

TABLE 5
Secondary efficacy outcomes (interim intent-to-treat set)

| | Surufatinib | | Placebo | | Hazard | | |
|---|---|---|---|---|---|---|---|
| | (N = 104) | | (N = 53) | | ratio | | P-value |
| Investigator-assessed outcomes | | | | | | | |
| Best overall response, n (%) | | | | | | | |
| Complete response | 0 | (0) | 0 | (0) | — | | — |
| Partial response* | 20 | (19.2) | 1 | (1.9) | — | | — |
| Stable disease | 64 | (61.5) | 34 | (64.2) | — | | — |
| Progressive disease | 8 | (7.7) | 16 | (30.2) | — | | — |
| Not evaluable | 12 | (11.5) | 2 | (3.8) | — | | — |
| Objective response rate, % (95% CI) | 19.2 | (12.2, 28.1) | 1.9 | (0.0, 10.1) | 12.4 | (1.8, 522.8) | 0.0021 ^{[1]} |
| Disease control rate, % (95% CI) | 80.8 | (71.9, 87.8) | 66.0 | (51.7, 78.5) | 2.1 | (0.9, 4.8) | 0.0774 ^{[2]} |
| Time to response, months (95% CI) | 3.8 | (2.3, 7.3) | 7.4 | (—, —) | — | | — |
| Duration of response, months (95% CI) | 7.4 | (3.7, —) | | — | — | | — |
| BIIRC-assessed outcomes | | | | | | | |
| Best overall response, n (%) | | | | | | | |
| Complete response | 0 | (0) | 0 | (0) | — | | — |
| Partial response* | 15 | (14.4) | 1 | (1.9) | — | | — |
| Stable disease | 73 | (70.2) | 35 | (66.0) | — | | — |
| Non-complete response, non- | 0 | | 1 | (1.9) | — | | — |
| progressive disease | | | | | | | |
| Progressive disease | 5 | (4.8) | 14 | (26.4) | — | | — |
| Not evaluable | 11 | (10.6) | 2 | (3.8) | — | | — |
| Objective response rate, % (95% CI) | 14.4 | (8.3, 22.7) | 1.9 | (0.0, 10.1) | 8.8 | (1.3, 375.9) | 0.0123 ^{[1]} |
| Disease control rate, % (95% CI) | 84.6 | (76.2, 90.9) | 67.9 | (53.7, 80.1) | 2.5 | (1.1, 6.1) | 0.0311 ^{[2]} |

| | | | | | | |
|---|---|---|---|---|---|---|
| Time to response, months (95% CI) | 3.9 | (1.9, 5.6) | 33.1 | (—, —) | — | — |

| | | | | | |
|---|---|---|---|---|---|
| Duration of response, months (95% CI) | 15.2 | (5.6, —) | — | — | — |

Secondary efficacy outcomes listed were performed in the interim intent-to-treat set.
*7 PRs were not confirmed by investigator assessment and 4 PRs were not confirmed by BIIRC assessment. BIIRC = Blinded Independent Image Review Committee. CI = confidence interval.
^{[1]} Fisher's exact test;
^{[2]} Cochran-Mantel-Haenszel (CMH) exact test.

Safety

In general, surufatinib was generally well tolerated in this study and the safety profile consistent with that previously reported for surufatinib (see, US. Patent Pub. No. 2019/0276439A1 and clinicaltrials.gov Identifier No. NCT02133157 for Phase I trial; and clinicaltrials.gov Identifier No. NCT02267967 for Phase Ib/II trial). Each of those documents are incorporated herein by reference in their entirety.

Most patients experienced at least one treatment-emergent adverse event (108 [95.6%] in the surufatinib group and 54 [91.5%] in the placebo group), primarily of grade 1 or 2. Common treatment-related adverse events are presented in Table 6. The most frequently reported (23% of patients) treatment-related grade 3 or higher adverse events for the surufatinib group versus the placebo group, respectively, were hypertension (43 [38.1%] versus 4 [6.8%]), proteinuria (11 [9.7%] versus 1 [1.7%]), hypertriglyceridaemia (8 [7.1%] versus 0) and diarrhea (5 [4.4%] versus 1 [1.7%]).

TABLE 6
Treatment-related adverse events of any grade in ≥10% patients (safety set)

| | Surufatinib | | | | Placebo | | | |
|---|---|---|---|---|---|---|---|---|
| | (N = 113) | | | | (N = 59) | | | |
| | Any grade | | ≥Grade 3 | | Any grade | | ≥Grade 3 | |
| Treatment-related adverse event, n | 107 | (94.7) | 76 | (67.3) | 51 | (86.4) | 16 | (27.1) |
| (%) | | | | | | | | |
| Proteinuria | 73 | (64.6) | 11 | (9.7) | 28 | (47.5) | 1 | (1.7) |
| Hypertension | 73 | (64.6) | 43 | (38.1) | 11 | (18.6) | 4 | (6.8) |
| Diarrhea | 58 | (51.3) | 5 | (4.4) | 15 | (25.4) | 1 | (1.7) |
| Blood thyroid stimulating hormone | 47 | (41.6) | 0 | | 6 | (10.2) | 0 | |
| increased | | | | | | | | |
| Blood bilirubin increased | 42 | (37.2) | 2 | (1.8) | 11 | (18.6) | 0 | |
| Hypertriglyceridaemia | 42 | (37.2) | 8 | (7.1) | 5 | (8.5) | 0 | |
| Aspartate aminotransferase increased | 27 | (23.9) | 2 | (1.8) | 19 | (32.2) | 1 | (1.7) |
| Occult blood positive | 27 | (23.9) | 0 | | 13 | (22.0) | 0 | |
| Abdominal pain | 26 | (23.0) | 2 | (1.8) | 5 | (8.5) | 0 | |
| Hyperuricaemia | 22 | (19.5) | 2 | (1.8) | 1 | (1.7) | 0 | |
| Hypoalbuminaemia | 21 | (18.6) | 0 | | 7 | (11.9) | 0 | |
| Alanine aminotransferase increased | 20 | (17.7) | 3 | (2.7) | 13 | (22.0) | 3 | (5.1) |
| Asthenia | 20 | (17.7) | 1 | (0.9) | 3 | (5.1) | 0 | |
| Abdominal distension | 19 | (16.8) | 2 | (1.8) | 7 | (11.9) | 0 | |
| Platelet count decreased | 18 | (15.9) | 2 | (1.8) | 0 | | 0 | |
| Hyperbilirubinaemia | 17 | (15.0) | 0 | | 0 | | 0 | |
| Vomiting | 16 | (14.2) | 3 | (2.7) | 8 | (13.6) | 1 | (1.7) |

| Nausea | 16 | (14.2) | 0 | | 7 | (11.9) | 0 |
|---|---|---|---|---|---|---|---|
| Hyperglycaemia | 16 | (14.2) | 2 | (1.8) | 5 | (8.5) | 0 |
| Headache | 16 | (14.2) | 0 | | 1 | (1.7) | 0 |
| Abdominal pain upper | 15 | (13.3) | 2 | (1.8) | 4 | (6.8) | 0 |
| Blood urine present | 15 | (13.3) | 1 | (0.9) | 4 | (6.8) | 0 |
| Fatigue | 15 | (13.3) | 0 | | 2 | (3.4) | 0 |
| Oedema peripheral | 15 | (13.3) | 2 | (1.8) | 1 | (1.7) | 0 |
| Dizziness | 14 | (12.4) | 0 | | 4 | (6.8) | 0 |
| Back pain | 14 | (12.4) | 0 | | 3 | (5.1) | 0 |
| Arthralgia | 14 | (12.4) | 0 | | 1 | (1.7) | 0 |
| Blood creatinine increased | 14 | (12.4) | 0 | | 1 | (1.7) | 0 |
| Hypercholesterolaemia | 14 | (12.4) | 0 | | 1 | (1.7) | 0 |
| Blood lactate dehydrogenase increased | 13 | (11.5) | 0 | | 3 | (5.1) | 0 |
| Protein urine present | 13 | (11.5) | 2 | (1.8) | 3 | (5.1) | 0 |

| Bilirubin conjugated increased | 13 | (11.5) | 0 | 1 | (1.7) | 0 |
|---|---|---|---|---|---|---|
| Hypothyroidism | 13 | (11.5) | 0 | 0 | | 0 |
| Decreased appetite | 12 | (10.6) | 0 | 3 | (5.1) | 0 |

| | | | | | |
|---|---|---|---|---|---|
| Haemoglobin increased | 12 | (10.6) | 0 | 0 | 0 |

Dose interruption due to adverse events, regardless of causality, occurred in 51 patients (45.1%) in the surufatinib group versus 14 patients (23.7%) in the placebo group, and dose reduction in 44 patients (38.9%) versus 3 patients (5.1%), respectively. The most common adverse events resulting in dose interruption or reduction were proteinuria (25 [22.1%] in the surufatinib group versus 1 [1.7%] in the placebo group) and hypertension (16 [14.2%] versus 1 [1.7%]). Discontinuation due to adverse events, regardless of causality, occurred in 12 patients (10.6%) in the surufatinib group and 4 patients (6.8%) in the placebo group. Adverse events leading to dose discontinuation occurred in ≥2 patients was vomiting (2 [1.8%] versus 0), with the remaining events affecting one patient (0.9%) each.

Rates of on-treatment deaths were comparable between the two groups (three patients [2.7%] in the surufatinib group versus one patient [1.7%] in the placebo group). Two on-treatment deaths occurring in the surufatinib group were attributed to adverse events: one was due to gastrointestinal haemorrhage, which was possibly related to the study drug by investigator assessment, and the other was due to cerebral haemorrhage, which was unlikely related to the study drug by investigator assessment. One on-treatment death in each of the surufatinib and placebo group was attributed to disease progression.

### Discussion

In this randomized Phase III study, surufatinib significantly prolonged median progression-free survival (10.9 months) compared to placebo (3.7 months) in patients with advanced progressive well-differentiated pancreatic NETs, with a 51% risk reduction of disease progression. The progression-free survival improvement was supported by the BIIRC assessment (13.9 months with surufatinib versus 4.6 months with placebo, HR 0.34). The benefit of surufatinib was observed across major subgroups. For the secondary efficacy endpoints, surufatinib produced an objective response rate of 19.2% and disease control rate of 80.8% according to investigator assessments compared with 1.9% and 66.0%, respectively, for placebo. Based on the study data from the pre-planned interim analysis, the study was terminated by the recommendation from the IDMC, for superior efficacy of surufatinib.

In the past decade, the combination of improved diagnostic testing leading to earlier disease diagnosis and the availability of new therapies have led to improved survival of patients with well-differentiated pancreatic NETs globally. Nevertheless, there is an existing unmet medical need, as most of these patients eventually die of their underlying malignancy. In SANET-p trial, the disease characteristics of the enrolled patients indicate more aggressive underlying disease, with an anticipated worse prognosis, when compared to the disease characteristics of pancreatic NETs patients from prior studies. Most patients with advanced pancreatic NETs enrolled in this study were of pathological grade 2 (87.6% in the surufatinib group vs 84.7% in the placebo group), with heavy tumor burden (95.6% versus 91.5% with liver metastasis and 54.9% versus 42.4% with three or more organs involved) and most patients had received prior treatment for advanced disease (65.5% versus 66.1%). SANET-p trial provided evidence for the effectiveness of surufatinib in these patients with advanced progressive well-differentiated pancreatic NETs, which supported surufatinib as an alternative treatment option in this population.

It is believed that the potent anti-tumor activity of surufatinib was attributed to its unique mechanism of target inhibition. Previous studies suggest that CSF-1-mediated signaling and TAMs play active roles in tumor progression; increased tumor-infiltrating macrophages correlated with higher tumor grade, liver metastasis, and greater tumor burden in human pancreatic NETs. Hence, CSF-1 signaling may have played a significant role in the progression of the underlying disease in the SANET-p population, as most of the enrolled patients had higher grade tumors or large tumor burden. Therefore, simultaneous inhibition of VEGF/FGF/CSF1 signaling by surufatinib may be advantageous for better disease control among these patients. In fact, not only in pancreatic NETs population, but also in a similarly progressive, heavily tumor-burdened, advanced well-differentiated extra-pancreatic NETs population, surufatinib has demonstrated significant progression-free survival prolongation in a previously reported parallel Phase III study (SANET-ep).

In the SANET-p trial, the safety profile of surufatinib was consistent with that in prior clinical studies of surufatinib. Most related adverse events were manageable through dose interruption or modification. Surufatinib treatment was well-tolerated for most patients, with a similar incidence of treatment discontinuation due to adverse events compared with that of the placebo group. Furthermore, patient reported outcomes remained generally comparable in surufatinib treated patients compared to placebo. This is an important finding, given palliation is the major focus of treatment in patients with incurable NETs.

Although this trial was conducted fully in a Chinese patient population, the results appear to be applicable to Western patients. Encouraging results have been reported from an ongoing phase 1 study of surufatinib in patients from the United States with heavily treated advanced pancreatic NETs (4 median prior lines of therapy), with an objective response rate of 25.0% (4/16: 3 confirmed partial response and 1 unconfirmed partial response). The safety profile observed in this Western patient population was similar to those observed in the SANET-p trial, as well as in other clinical studies of surufatinib in NETs patients.

Surufatinib demonstrated statistically significant and clinically meaningful efficacy compared with placebo and was well-tolerated in patients with advanced, progressive, well-differentiated pancreatic NETs.

### Example 2: SANET-Ep Trial

1. Summary of Phase III Clinical Study (SANET-Ep Trial)

SANET-ep (NCT02588170) is a randomized, double-blind, placebo controlled, multi-center Phase III clinical study to evaluate the efficacy and safety of surufatinib compared to placebo in patients with well-differentiated, progressive, advanced (unresectable local progression or distant metastasis) extra-pancreatic neuroendocrine tumors.

2. Study Design and Dose Administration

This study is a randomized, double-blind, placebo-controlled, multi-center Phase III study to assess the efficacy and safety of Surufatinib in treating advanced extra-pancreatic neuroendocrine tumors.

The targeted population are progressive, locally advanced or distant metastatic extra-pancreatic NETs (including, but not limited to: lungs, thymus gland and gastrointestinal origins like stomach, duodenum, liver, jejunum, ileum, colon, caecum, vermiform appendix and rectum, and unknown origin) patients with no possibility of radical resection of tumor, and of pathologically with low or intermediate grade (G1 or G2). For Gastrointestinal neuroendocrine tumors (GI-NETs), G1 is defined as <2 mitoses/10 high-power field

[HPF] and/or <3% Ki-67 index; G2 is defined as 2-20/10 HPF and/or 3-20% Ki-67 index; for NETs originating from the lung and thymus gland, G1 is defined as <2 mitoses/10 HPF and no necrosis; G2 is defined as 2-10/10 HPF and/or foci of necrosis. NETs from origin other than GI-NET, lung and thymus, or from unknown origins should be graded according to the GI-NET grading criteria. If the mitotic ratio and Ki-67 index correspond to different grade, the higher grade should be used to assign classification. All patients must provide tumor samples for central pathology review to check as of whether the diagnostics and grade score comply with inclusion criteria. Patients must have radiological documentation of progression of disease within 12 months prior to randomization. Patients must have previously progressed no more than two types of systemic anti-tumor therapy, including long-acting somatostatin analogs (SSAs), interferon, PRRT (peptide receptor radionuclide therapy), mammalian target of rapamycin (mTOR) inhibitors or chemotherapy (all chemotherapies are deemed as a single therapy regardless of drug or time); patients who are unable or unwilling to receive such treatments are also eligible if all other criteria are met.

Double-Blinded Stage

Approximately 273 patients will be randomly assigned (in 2:1 ratio) to the following treatment groups based on interactive web response system IWRS:

Surufatinib, 300 mg, once daily (QD), orally

Placebo, 300 mg, once daily (QD), orally

Continuous administration (every 4 weeks as a treatment cycle). Surufatinib can be taken with or without food and should be taken as a single administration per day. It is recommended to take the drug at the same time every day. If the patient vomits after dosing surufatinib, there is no need to make up. If a dosage is missed, do not make up the missed dose in the next day, and take the next dose as scheduled.

During the treatment, physician should closely monitor the patients and adjust dosage according to the safety and tolerability of individual patients, including dose interruption, dose reduction or permanently discontinue treatment. The dose adjustment should follow the guidance of interruption first and then dose reduction.

After dose interruption, if the adverse reaction resolves to ≤Grade 1 within one week then the original dose is reinitiated. If the adverse reaction resolves to ≤Grade 1 within 4 weeks, it is recommended to adjust the dose under the instruction of the physician:

the first dose is adjusted to 250 mg QD (5 capsules, each capsule containing 50 mg of surufatinib);

the second dose is adjusted to 200 mg QD (4 capsules, each capsule containing 50 mg of surufatinib); if the dose of 200 mg QD is still intolerable, then the dose of 200 mg QD for 3 weeks on and 1 week off or permanently discontinue treatment should be considered.

The general guidance of dose modification is shown in Table 7 and Table 8.

TABLE 7
Dose Adjustment for Hematological Toxicity

| NCI CTCAE v4.03 Toxicity Grading | Action |
|---|---|
| Grade 1 or 2 | None |
| Grade 3 or 4^{b} | |
| Manageable/reversible after reduction | Withhold^{a} |
| Grade 3 or 4 decrease | Reduce to a lower dose level |
| to ≤Grade 1 or baseline | |
| level within 28 days | |
| Reoccurrence of Grade 3 toxicity | Reduce 1 dose level or |
| | discontinue treatment |
| Reoccurrence of Grade 4 toxicity | Discontinue treatment |
| Grade 3 or 4 | |
| Unmanageable/irreversible | Discontinue treatment |
| after reduction | |

^{a}Treatment is to be withheld until toxicity returns to ≤grade 1 or baseline level. Patients whose toxicity has been reduced for over 28 days can generally be discontinued from the study, unless the investigator determines that the patients can still benefit clinically from the study. In this case, the investigator is to discuss with the sponsor as to whether to keep the patient in the study or not.
^{b}If grade 3 or grade 4 leukocyte or neutrophils decrease and recover to ≤grade 1 or to baseline level within 28 days, administer the original dose. Only complicated neutropenia, including recurrent or persistent grade 4 neutropenia occurring for >7 days or concurrent with a fever or infection will require dose reduction.

TABLE 8
Dose Adjustment for Hon-Hematological Toxicity

| NCI CTCAE v4.03 Toxicity Grading | Action |
|---|---|
| Grade 1 or 2 (Tolerable) | None |
| Grade 2 (intolerable) ^{d} | Withhold ^{c} |
| Toxicity decrease | Maintain previous dose |
| to ≤Grade 1 or baseline | |
| level within 28 days | |
| Recurrent | Reduce 1 dose level |
| Grade 3 or 4 ^{d} | |
| Manageable/reversible after reduction | Withhold ^{c} |
| Grade 3 toxicity decrease | Reduce 1 dose level |
| to ≤Grade 1 within | |
| 28 days^{e} | |
| Reoccurrence of Grade 3/4 toxicity | Reduce 1 dose level or |
| | discontinue treatment* |
| Grade 3 or 4 | |
| Unmanageable/irreversible after reduction | Discontinue treatment |

^{c} Patients whose toxicity has been persistent without recovery for over 28 days can generally be discontinued from the study, unless the investigator determines that the patients can still benefit clinically from the study. In this case, the investigator is to discuss with the sponsor as to whether to keep the patient in the study.
^{d} For nausea, vomiting or diarrhea, supportive treatment is to be given first. If after the supportive treatment, the investigational product is still not tolerated, study drug is to be withheld until the toxicity decreases to ≤grade 1 or baseline level. For toxic events that the investigator considers unlikely to be serious or life threatening and that can be tolerated by patients, such as hyperuricemia, hypophosphatemia and alopecia laboratory results, the investigational treatment may stay at the same dose level without dose reduction, provided supportive care has been given to treat the event.
*The investigator is to decide whether to use this administration method or not according to the patient's condition and tolerability.

Also, during the treatment, physician could closely monitor the patients and adjust dosage according to the proteinuria level of the patient, including no dose adjustment, dose interruption, dose reduction or permanently discontinuation.

When the patient meets one or more criteria chosen from: when urinalysis shows protein+ and 24-hour urine protein quantity is less than 1.0 g, no dose adjustment is required. When urinalysis shows protein 2+ or 3+ and 24-hour urine protein quantity is 1.0-2.0 g, excluding 2.0 g, the previous dose can be retained to continue the treatment. If 24-hour urine protein quantity ≥2.0 g, and one or more criteria below is met, dose adjustment, dose reduction or dose discontinuation may be needed:

when a first 24-hour urine protein quantity ≥2.0 g occurs, a dose interruption applies, and the dose of surufatinib is reduced to 250 mg if the test results resolve to ≤Grade 1 within 4 weeks;

when a second 24-hour urine protein quantity ≥2.0 g occurs, the dose interruption applies, and the dose of surufatinib is reduced to 200 mg if the test results resolve to ≤Grade 1 within 4 weeks; and

when a third 24-hour urine protein quantity ≥2.0 g occurs, the dose interruption applies, and the dose of surufatinib is reduced to 200 mg with 3 weeks on and 1 week off if the test results resolve to ≤Grade 1 within 4 weeks, or dose discontinuation applies.

The common guidance of dose modification for proteinuria is shown in Table 9.

TABLE 9
Dose Modification for Proteinuria

| CTCAE Grade | | Dose Modification | |
|---|---|---|---|
| Grade 1 | urinalysis shows protein+; | No dose adjustment required | |
| | 24-hour urine protein | | |
| | quantity <1.0 g | | |
| Grade 2 | urinalysis shows protein | Previous dose can be retained to continue the | |
| | 2+ or 3+; 24-hour urine | treatment. | |
| | protein quantity 1.0-2.0 g | | |
| | (excluding 2.0 g) | | |
| | | First | Drug interruption; |
| | | occurrence | Reduce the dose to 250 mg if the |
| | | | test results resolve to ≤Grade 1 |
| | | | within 4 weeks. |
| Grade 2~3 | 24-hour urine protein | Second | Drug interruption; |
| | quantity ≥2.0 g | occurrence | Reduce the dose to 200 mg if the |
| | | | test results resolve to ≤Grade 1 |
| | | | within 4 weeks. |
| | | Third | Drug interruption; |
| | | occurrence | Reduce the dose to 200 mg with 3 |
| | | | weeks on and 1 week off, if the test |
| | | | results resolve to ≤Grade 1 within 4 |
| | | | weeks. Or consider drug |
| | | | discontinuation according to the |
| | | | physician's decision. |

The severity of adverse reactions was graded by the National Cancer Institute Common Terminology Criteria for Adverse Events version 4.03 (NCI CTCAE v4.03).

Randomized stratification factors are: NET pathological grade (GI or G2), previous systemic antineoplastic therapy (yes or no), primary tumor site: A (jejunum, ileum, duodenum, thymus gland, caecum), or B (lung, stomach, liver, vermiform appendix, colon, rectum), or C (other origin or unknown origin).

According to the randomized result, the patients will receive continuous oral treatment, every 4-week treatment cycle until progression of disease occurs, intolerable toxicity or other protocol-specified end-of-treatment criteria is met.

Other antitumor therapies are forbidden during the treatment. However, when patients have obvious NET functional symptoms and need to use somatostatin analogues (SSAs) to control symptoms, they can receive short-term SSAs treatment.

In accordance with RECIST 1.1, the tumor should be assessed every 8 weeks (±3 days) within the first year and every 12 weeks (±3 days) after the patient has been treated for more than one year.

Inclusion Criteria

The enrolled participants should meet all following criteria:

1. Adequately understand the study and voluntarily sign the Informed Consent Form;

2. Be at least 18 years old;

3. Based on central pathology review results, patients have a confirmed histologically pathology diagnosis of low- or intermediate grade (G1 or G2) advanced (unresectable or distant metastatic) extra-pancreatic NETs with origins including, but not limited to, the lung, thymus, the gastrointestinal tract (stomach, duodenum, liver, jejunum, ileum, colon, cecum, appendix, rectum) and unknown origin etc. For Gastrointestinal neuroendocrine tumors (GI-NETs), G1 is defined as <2 mitoses/10 high-power field (HPF) and/or <3% Ki-67 index; G2 is defined as 2-20/10 HPF and/or 3-20% Ki-67 index. For NETs originating from the lung and thymus gland, G1 is defined as <2 mitoses/10 HPF and no necrosis; G2 is defined as 2-10/10 HPF and/or foci of necrosis. NETs from origin other than GI-NET, lung and thymus, or from unknown origins should be graded according to the GI-NET grading criteria. If the mitotic ratio and Ki-67 index correspond to different grade, the higher grade should be used to assign classification.

4. Have previously received no more than two types of systemic anti-tumor therapy, including long-acting somatostatin analogs (SSAs), interferon, peptide receptor radionuclide therapy, mammalian target of rapamycin (mTOR) inhibitors or chemotherapy (all chemotherapies are deemed as a single therapy regardless of drug or time); patients who are unable or unwilling to receive such treatments are also eligible.

5. Patients must have radiological documentation of progression of disease within 12 months prior to randomization.

6. Have measurable lesions (according to RECIST 1.1).

7. Absolute neutrophil count (ANC) of ≥1.5×10⁹/L, platelet count of 2 100×10⁹/L, and hemoglobin ≥9 g/dL;

8. Serum total bilirubin <1.5 times the upper limit of normal (ULN);

9. Patients who do not have liver metastasis, with alanine minotransferase (ALT), aspartate aminotransferase (AST) levels ≤2.5 times the ULN; and who do have liver metastatic, with ALT and AST ≤5 times ULN.

10. Serum creatinine <1.5 times ULN and creatinine clearance ≥60 ml/min;

11. International Normalized Ratio (INR) ≤1.5 ULN and activated partial thromboplastin time (APTT) ≤1.5 ULN.

12. Have a performance status (PS) of 0 or 1 on the Eastern Cooperative Oncology Group (ECOG) scale;

13. Have expected survival of more than 12 weeks;

14. Male or female patients with reproductive potential must agree to use an effective contraceptive method, for example, double-barrier device, condom, oral or injected birth control medication or intrauterine device, during the study and within 90 days after study treatment discontinuation. All female patients are considered to be fertile, unless the patient had natural menopause or artificial menopause or sterilization (e.g. hysterectomy, bilateral adnexectomy or radioactive ovarian irradiation).

Exclusion Criteria

Enrolled participants will be excluded for any one of the below criteria:

1. High grade (G3) neuroendocrine cancer, adenocarcinoid, pancreatic islet cell carcinoma, goblet cell carcinoid, large cell neuroendocrine carcinoma and small cell carcinoma;

2. Neuroendocrine tumors with pancreatic origins;

3. Functional NETs which need to be treated with long acting SSAs to control disease related syndromes, such as insulinoma, gastrinoma, glucagonoma, somatostatinoma, ACTHoma, VIPoma, accompanied by carcinoid syndrome, Zollinger-Ellison syndrome or other active symptoms;

4. Have received anti-vascular endothelial growth factor (VEGF/VEGFR) targeted drugs and progressed upon these drugs;

5. Urinalysis shows urine protein ≥2+ or 24-hour protein quantity test shows urinary protein ≥1 g;

6. Serum potassium, calcium (albumin-bound ionic or corrected) or magnesium exceed the normal range with clinical significance;

7. Under anti-hypertension treatment, still uncontrolled hypertension, defined as: systolic blood pressure >140 mmHg or diastolic blood pressure >90 mmHg;

8. Gastrointestinal disease or condition that investigators suspect may affect drug absorption, including, but not limited to, active gastric and duodenal ulcers, ulcerative colitis and other digestive disease, gastrointestinal tumor with active bleeding, or other gastrointestinal conditions that may cause bleeding or perforation at investigators discretion:

9. History or presence of a serious hemorrhage (>30 ml within 3 months), hemoptysis (>5 ml blood within 4 weeks) or a thromboembolic event (including transient ischemic attack) within 12 months;

10. Clinically significant cardiovascular disease, including but not limited to, acute myocardial infarction within 6 months prior to enrollment, severe/unstable angina pectoris or coronary artery bypass grafting, congestive heart failure according to the New York Heart Association (NYHA) classification ≥2; ventricular arrhythmias which needs drug treatment; LVEF (LVEF) <50%;

11. Mean corrected QT interval (QTc) ≥480 msec;

12. Other malignancies diagnosed within the previous 5 years, except basal cell carcinoma, squamous-cell carcinoma or in-situ cervical carcinoma after radical resection;

13. Anti-tumor therapy received within 4 weeks prior to the initiation of the investigational treatment, including, but not limited to, chemotherapy, radical radiotherapy, targeted therapy, immunotherapy and anti-tumor Chinese medicine treatment, hepatic chemoembolization, cryoablation and radiofrequency ablation;

14. Palliative radiotherapy for a bone metastasis lesion within 2 weeks prior to the initiation of the investigational treatment;

15. Drugs containing St John's wort taken within 3 weeks prior to the first study treatment, or other strong inducers with Cytochrome P450 3A4 (CYP3A4) or strong inhibitors taken within two weeks prior to the first study treatment;

16. Any clinically significant active infection, including, but not limited to, human immunodeficiency virus (HIV) infection;

17. History of clinically significant hepatic disease, including, but not limited to, known hepatitis B virus (HBV) infection with HBV DNA positive (copies ≥1×10⁴/m); known Hepatitis C virus HCV) infection with HCV RNA positive (copies ≥1 ×10³/ml); or liver cirrhosis, etc.

18. Surgery (except biopsy) within 28 days prior to the initiation of investigational treatment or unhealed surgical incision;

19. Brain metastases and/or spinal cord compression not treated by surgery and/or radiotherapy, and with no clinical imaging evidence of disease stability;

20. Toxicity from a previous anti-tumor treatment that does not return to Grade 0 or 1 (except for hair loss);

21. Received investigational treatments in other clinical studies within 4 weeks prior to enrollment;

22. Women who are pregnant or lactating;

23. Other disease, metabolic disorder, physical examination anomaly, abnormal laboratory result, or any other conditions are inappropriate for the use of the investigational product or affect interpretation of study results.

Endpoints

Therapeutic Endpoints:

1) Primary therapeutic endpoint: PFS (according to RECIST 1.1 criteria)

2) Secondary therapeutic endpoint: Objective Response Rate of the tumor (ORR), Disease Control Rate (DCR), Duration of Response (DoR), Time to Response (TTR) and Overall Survival (OS) (according to RECIST 1.1 criteria)

Exploratory Endpoints:

For patients whose serum CgA are abnormally elevated from two arms, the serum CgA and 24 h urine 5-HIAA level change of patients after treatment was compared with baseline.

The change of VEGF, FGF23 and other target-related biomarkers after treatment in plasma and/or tumor tissues compared with baseline

To determine the correlation between steady-state pharmacokinetics in vivo in patients (blood drug concentration based on C1D15 data, including but not limited to Cₘₐₓ, Cₘᵢₙ and AUCₛₛ) and tumor response, serum CgA level as well as AE incidence.

The score change of life quality questionnaire EORTC QLO-C30 and QLQ-GI.NET 21 from two arms after treatment were compared with baseline.

Results and Analysis

Patients and Treatments

As of the cut-off date (Mar. 31, 2019), a total of 198 patients were randomly enrolled in a 2:1 ratio into the study cohorts (129 patients in surufatinib cohort, 69 patients in placebo cohort).

The main baseline characteristics of the patients is shown in Table 10. The demographic data and baseline characteristics of the placebo cohort were generally consistent with those of the Surufatinib cohort.

TABLE 10
Demographic and Baseline Clinical Characteristics (Intent-to-treatment Population)

| | Surufatinib | | Placebo | |
|---|---|---|---|---|
| Characteristic | (n = 129) | | (n = 69) | |
| Age in year, median (range) | 52.0 | (19.0-72.0) | 54.0 | (25.0-79.0) |
| Male, n (%) | 73 | (56.6) | 35 | (50.7) |
| ECOG performance status, n (%) | | | | |
| 0 | 72 | (55.8) | 46 | (66.7) |
| 1 | 57 | (44.2) | 23 | (33.3) |
| Primary tumor location, n (%) | | | | |
| Gastrointestinal tract | 61 | (47.3) | 32 | (46.4) |
| Lung | 12 | (9.3) | 11 | (15.9) |
| Others | 38 | (29.4) | 17 | (24.7) |
| Unknown | 18 | (14.0) | 9 | (13.0) |
| Pathologic grade, n (%) | | | | |
| Grade 1 | 21 | (16.3) | 11 | (15.9) |
| Grade 2 | 108 | (83.7) | 58 | (84.1) |
| Function status | | | | |
| Functional | 5 | (3.9) | 2 | (2.9) |
| Non-functional | 122 | (94.6) | 67 | (97.1) |
| Unknown | 2 | (1.6) | 0 | |
| Metastatic sites, n (%) | | | | |
| Liver | 97 | (75.2) | 53 | (76.8) |
| Lymph nodes | 61 | (47.3) | 33 | (47.8) |
| Lung | 33 | (25.6) | 18 | (26.1) |
| Bone | 40 | (31.0) | 26 | (37.7) |
| Other | 49 | (38.0) | 26 | (37.7) |
| Number of organs involved, n (%) | | | | |
| ≤2 | 43 | (33.3) | 25 | (36.2) |
| ≥3 | 86 | (66.7) | 44 | (63.8) |
| Time from disease progression to randomization, n (%) | | | | |
| ≤3 months | 114 | (88.4) | 58 | (84.1) |
| >3 to ≤6 months | 12 | (9.3) | 9 | (13) |
| >6 to ≤12 months | 3 | (2.3) | 2 | (2.9) |
| Prior systemic antitumor drug for advanced disease, n (%) | 89 | (69.0) | 44 | (63.8) |
| Prior everolimus treatment | 10 | (7.8) | 8 | (11.6) |
| Prior SSA treatment | 44 | (34.1) | 19 | (27.5) |
| Prior systemic chemotherapy | 52 | (40.3) | 27 | (39.1) |

The intent-to-treatment population which included all patients randomized.
ECOG = European Co-operative Oncology Group. SSA = somatostatin analog.

The median treatment duration of surufatinib and placebo was 217 days (range 4.0 to 1032.0) and 146 days (range 6.0 to 844.0), respectively. The mean relative dose intensity (the ratio of actual to planned dose intensity) was 86.4% (standard deviation 13.2%) and 96.8% (standard deviation 9.0%) in the surufatinib and placebo group separately.

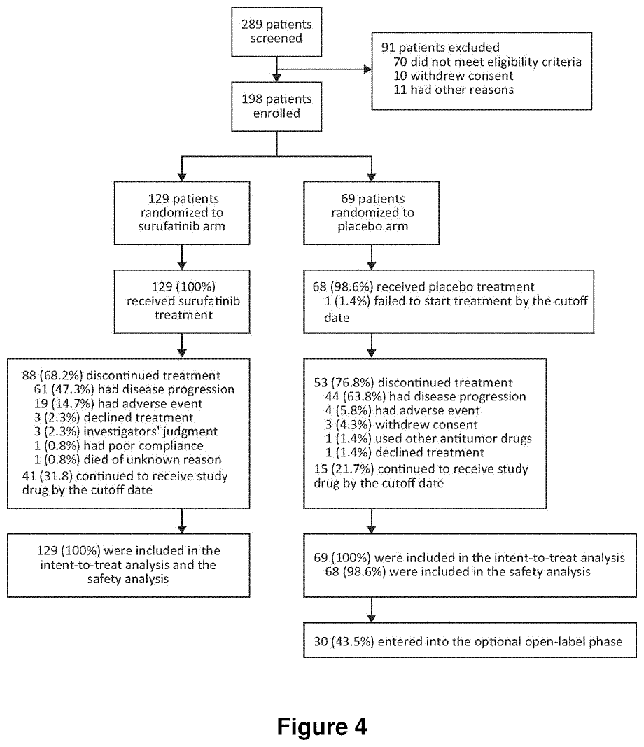

At the time of data cut-off, 88 patients (68.2%) and 53 (76.8%) in surufatinib and placebo group had discontinued double-blinded study treatment respectively (see FIG. 4). The primary reasons for treatment discontinuation were disease progression (47.3% for surufatinib vs. 63.8% for placebo) and adverse events (14.7% vs. 5.8%).

Efficacy

At the time of data cutoff (31 Mar. 2019), 128 progression-free survival events had occurred (65% maturity) as assessed by investigators. The median follow-up for progression-free survival was 13.8 months in the surufatinib group and 16.6 months in the placebo group. The median progression-free survival as assessed by the investigators was 9.2 months (95% confidence interval [CI], 7.4 to 11.1) in the surufatinib group versus 3.8 months (95% CI, 3.7 to 5.7) in the placebo group (HR, 0.33; 95% CI, 0.22, 0.50; P<0.001).

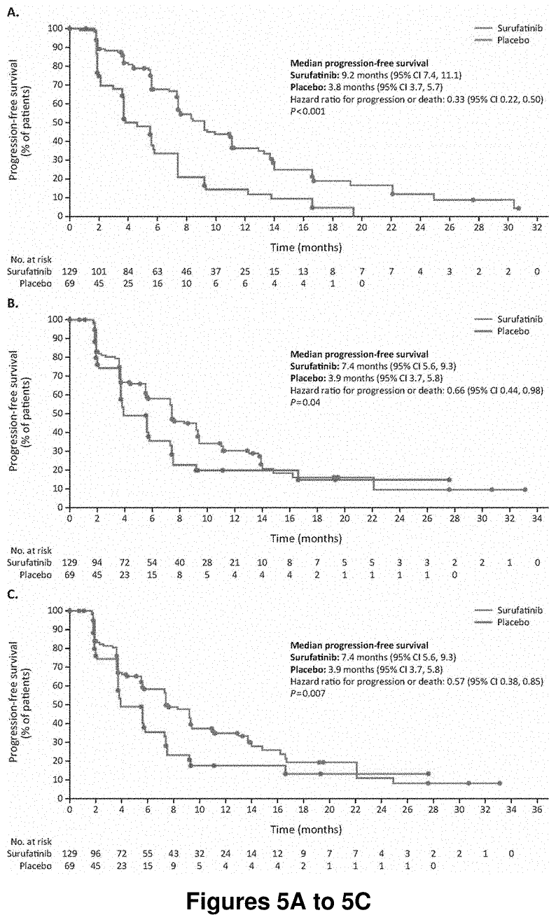

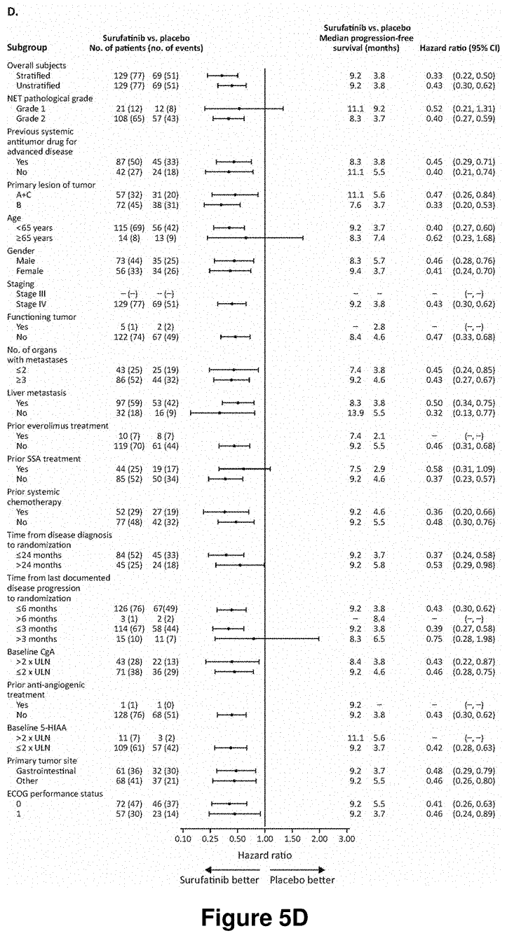

Overall, surufatinib reduced the risk of disease progression or death by 67% compared with placebo (see FIG. 5A). Prolonged progression-free survival with surufatinib was also noted in the BIIRC-assessment sensitivity analysis, with median progression-free survival of 7.4 months (95% CI, 5.6, 9.3) versus 3.9 months (95% CI, 3.7, 5.8) respectively (HR, 0.66; 95% CI, 0.44, 0.98; P=0.037) (see FIG. 5B). An independent, blinded, post-hoc image adjudication was performed for 35 patients. Based on the adjudicated BIIRC results, the HR of progression-free survival was 0.57 (95% CI, 0.38, 0.85; P=0.007) (see FIG. 5C). The progression-free survival benefit, as assessed by investigators, favored surufatinib treatment across all subgroups (see FIG. 5D).

FIG. 5A shows Kaplan-Meier curves of progression-free survival as assessed by investigators. Events of progression or death were observed in 77 patients (59.7%) in the surufatinib group and 51 (73.9%) in the placebo group. FIG. 5B shows progression-free survival as assessed by Blinded Independent Image Review Committee (BIIRC). Events of progression or death were observed in 80 patients (62.0%) in the surufatinib group and 41 (59.4%) in the placebo group. FIG. 5C shows progression-free survival based on the post-hoc adjudicated BIIRC assessment. Events of progression or death were observed in 80 patients (62.0%) in the surufatinib group and 42 (60.9%) in the placebo group. A forest plot of subgroup analyses based on investigators' assessment is shown in FIG. 5D.

In the interim intent-to-treat set, 13 patients in the surufatinib group and no patient in the placebo group achieved partial response, as assessed by the investigators, resulting in objective response rates of 10.3% (95% CI, 5.6, 17.0) versus 0% (P=0.005), respectively. The median time to response and median duration of response are shown in Table 10. Trends in overall response by BIIRC assessment were similar to those reported by the investigators (Table 11).

TABLE 11
Secondary Efficacy Endpoints (Interim Intent-to-treat Population).

| | | | |
|---|---|---|---|
| Surufatinib | Placebo | Hazard | |
| (N = 126) | (N = 64) | ratio | P-value |

Investigator-assessed

| Best overall response, n (%) | | | | | | | |
|---|---|---|---|---|---|---|---|
| Complete response | 0 | (0) | 0 | (0) | — | | — |
| Partial response | 13 | (10.3) | 0 | (0) | — | | — |
| Stable disease | 96 | (76.2) | 42 | (65.6) | — | | — |
| Progressive disease | 13 | (10.3) | 18 | (28.1) | — | | — |
| Not evaluable | 4 | (3.2) | 4 | (6.3) | — | | — |
| Objective response rate, % | 10.3 | (5.6, 17.0) | 0 | (0) | — | | 0.005^{[1]} |
| (95% CI) | | | | | | | |
| Disease control rate, % | 86.5 | (79.3, 91.9) | 65.6 | (52.7, 77.1) | 3.3 | (1.5, 7.3) | 0.002^{[2]} |
| (95% CI) | | | | | | | |

| | | | | | |
|---|---|---|---|---|---|
| Time to response, months | 3.7 | (1.8, 5.5) | — | — | — |
| (95% CI) | | | | | |
| Duration of response, | 5.6 | (2.0, 17.5) | — | — | — |
| months (95% Cl) | | | | | |

BIIRC-assessed

| Best overall response, n (%) | | | | | | | |
|---|---|---|---|---|---|---|---|
| Complete response | 0 | (0) | 0 | (0) | — | | — |
| Partial response* | 10 | (7.9) | 0 | (0) | — | | — |
| Stable disease | 88 | (69.8) | 43 | (67.2) | — | | — |
| Mon-complete response, | 1 | (0.8) | 2 | (3.1) | — | | — |
| non-progressive disease | | | | | | | |
| Progressive disease | 22 | (17.5) | 15 | (23.4) | — | | — |
| Not evaluable | 5 | (4.0) | 4 | (6.3) | — | | — |
| Objective response rate, % | 7.9 | (3.9, 14.1) | 0 | (0) | — | | 0.017^{[1]} |
| (95% CI) | | | | | | | |
| Disease control rate, % | 77.8 | (69.5, 84.7) | 67.2 | (54.3, 78.4) | 1.7 | (0.8, 3.5) | 0.187^{[2]} |
| (95% CI) | | | | | | | |

| | | | | | |
|---|---|---|---|---|---|
| Time to response, months | 2.9 | (1.7, 3.7) | — | — | — |
| (95% CI) | | | | | |
| Duration of response, | 5.6 | (1.8, —) | — | — | — |
| months (95% CI) | | | | | |

The interim intent-to-treatment population included all randomized patients who had at least one post-baseline disease assessment performed ≥6 weeks from first dose or discontinued double blind study treatment for any reason. Objective response rate defined as the proportion of patients whose best overall response was a complete or partial response, regardless of response confirmation; disease control rate defined as the proportion of patients with best overall response of complete response, partial response, or stable disease; time to response defined as the time from randomization to first documented response; duration of response defined as the time from first documented response to first documented disease progression or death due to any cause. Objective response rate and disease control rate were calculated by treatment group, along with corresponding exact two-sided 95% confidence intervals, using the Clopper-Pearson method.
^{[1]}Fisher's exact test;
^{[2]}Cochran-Mantel-Haenszel (CMH) exact test.
BIIRC = Blinded Independent Image Review Committee. CI = confidence interval.
*2 PRs were not confirmed.

Safety

In general, surufatinib was generally well tolerated in this study and the safety profile consistent with that previously reported for surufatinib (see, US. Patent Pub. No. 2019/0276439A1 and clinicaltrials.gov Identifier No. NCT02133157 for Phase I trial; and clinicaltrials.gov Identifier No. NCT02267967 for Phase Ib/II trial). Each of those documents are incorporated herein by reference in their entirety.

Almost all patients experienced at least one treatment-related adverse event (97.7% in the surufatinib group and 95.6% in the placebo group), primarily of grade 1 or 2. Common treatment-related adverse events are presented in Table 12. The most frequently reported (≥3% of patients) treatment-related grade 3 or higher adverse events were hypertension (36.4% with surufatinib vs. 13.2% with placebo), proteinuria (19.4% vs. 0%), anemia (4.7% vs. 2.9%), aspartate aminotransferase increased (3.9%), urine protein present and hyperbilirubinemia (3.9% vs. 0% for each event) and alanine aminotransferase increased (3.1% vs. 0%).

TABLE 12
Treatment-related Adverse Events (Safety Population)

| | Surufatinib | | | | Placebo | | | |
|---|---|---|---|---|---|---|---|---|
| | (N = 129) | | | | (N = 68)* | | | |
| | Any grade | | ≥Grade 3 | | Any grade | | ≥Grade 3 | |
| Treatment-related adverse event, n | 126 | (97.7) | 95 | (73.6) | 65 | (95.6) | 22 | (32.4) |
| (%) | | | | | | | | |
| Proteinuria | 88 | (68.2) | 25 | (19.4) | 33 | (48.5) | 0 | (0) |
| Hypertension | 83 | (64.3) | 47 | (36.4) | 18 | (26.5) | 9 | (13.2) |
| Diarrhea | 58 | (45.0) | 2 | (1.6) | 12 | (17.6) | 0 | (0) |
| TSH increased | 50 | (38.8) | 0 | (0) | 5 | (7.4) | 0 | (0) |
| Blood bilirubin increased | 49 | (38.0) | 0 | (0) | 12 | (17.6) | 0 | (0) |
| AST increased | 47 | (36.4) | 5 | (3.9) | 17 | (25.0) | 2 | (2.9) |
| Occult blood positive | 42 | (32.6) | 0 | (0) | 12 | (17.6) | 0 | (0) |
| Hypertriglyceridemia | 38 | (29.5) | 3 | (2.3) | 5 | (7.4) | 0 | (0) |
| ALT increased | 31 | (24.0) | 4 | (3.1) | 19 | (27.9) | 0 | (0) |
| Hyperbilirubinemia | 29 | (22.5) | 5 | (3.9) | 7 | (10.3) | 0 | (0) |
| Upper abdominal pain | 25 | (19.4) | 1 | (0.8) | 8 | (11.8) | 0 | (0) |
| Hypoalbuminemia | 24 | (18.6) | 0 | (0) | 3 | (4.4) | 0 | (0) |
| Peripheral edema | 23 | (17.8) | 0 | (0) | 2 | (2.9) | 0 | (0) |
| Hyperuricemia | 22 | (17.1) | 3 | (2.3) | 4 | (5.9) | 1 | (1.5) |
| Nausea | 22 | (17.1) | 0 | (0) | 8 | (11.8) | 0 | (0) |
| Abdominal pain | 21 | (16.3) | 2 | (1.6) | 5 | (7.4) | 0 | (0) |
| Platelet count decreased | 21 | (16.3) | 1 | (0.8) | 3 | (4.4) | 0 | (0) |
| Blood urine present | 20 | (15.5) | 0 | (0) | 5 | (7.4) | 0 | (0) |
| Weight decreased | 20 | (15.5) | 0 | (0) | 4 | (5.9) | 0 | (0) |
| Protein urine present | 20 | (15.5) | 5 | (3.9) | 3 | (4.4) | 0 | (0) |
| Anemia | 19 | (14.7) | 6 | (4.7) | 9 | (13.2) | 2 | (2.9) |
| Conjugated bilirubin increased | 19 | (14.7) | 0 | (0) | 7 | (10.3) | 0 | (0) |
| Blood creatinine increased | 19 | (14.7) | 0 | (0) | 2 | (2.9) | 0 | (0) |
| Abdominal distention | 19 | (14.7) | 0 | (0) | 4 | (5.9) | 0 | (0) |
| Vomiting | 19 | (14.7) | 0 | (0) | 4 | (5.9) | 0 | (0) |
| Electrocardiogram T wave | 18 | (14.0) | 0 | (0) | 3 | (4.4) | 0 | (0) |
| abnormal | | | | | | | | |
| Asthenia | 17 | (13.2) | 0 | (0) | 6 | (8.8) | 0 | (0) |
| Back pain | 17 | (13.2) | 1 | (0.8) | 9 | (13.2) | 0 | (0) |
| Constipation | 17 | (13.2) | 0 | (0) | 3 | (4.4) | 0 | (0) |
| Hypothyroidism | 17 | (13.2) | 0 | (0) | 0 | | 0 | (0) |
| Appetite decreased | 16 | (12.4) | 1 | (0.8) | 6 | (8.8) | 0 | (0) |
| Fatigue | 16 | (12.4) | 0 | (0) | 4 | (5.9) | 1 | (1.5) |
| Hypocalcemia | 15 | (11.6) | 1 | (0.8) | 7 | (10.3) | 0 | (0) |
| Face edema | 15 | (11.6) | 0 | (0) | 2 | (2.9) | 0 | (0) |
| White blood cell count decreased | 15 | (11.6) | 1 | (0.8) | 5 | (7.4) | 0 | (0) |
| Hemoglobin increased | 15 | (11.6) | 0 | (0) | 2 | (2.9) | 0 | (0) |
| Neutrophil count decreased | 15 | (11.6) | 1 | (0.8) | 1 | (1.5) | 0 | (0) |
| Electrocardiogram ST-T change | 14 | (10.9) | 0 | (0) | 4 | (5.9) | 0 | (0) |
| Dizziness | 14 | (10.9) | 1 | (0.8) | 5 | (7.4) | 0 | (0) |
| Headache | 14 | (10.9) | 1 | (0.8) | 5 | (7.4) | 1 | (1.5) |
| Red blood cells urine positive | 13 | (10.1) | 0 | (0) | 3 | (4.4) | 0 | (0) |
| White blood cells urine positive | 13 | (10.1) | 0 | (0) | 3 | (4.4) | 0 | (0) |
| Blood uric acid increased | 13 | (10.1) | 3 | (2.3) | 2 | (2.9) | 0 | (0) |

*Safety population included all patients who received at least one dose of a trial drug (surufatinib or placebo). One patient in the placebo group who was randomized but didn't start treatment was not included in the safety population. Treatment-related adverse events of any grade in ≥10% of patients are listed. ALT = alanine transaminase. AST = aspartate transaminase. TSH = thyroid stimulating hormone.

Dose interruption due to adverse events regardless of causality occurred in 62 (48.1%) patients in the surufatinib group versus 15 (22.1%) patients in the placebo group, and dose reduction in 62 (48.1%) versus 5 (7.4%), respectively. The most common adverse events regardless of causality resulting in dose interruption or reduction were proteinuria (29.5% with surufatinib vs. 1.5% with placebo) and hypertension (15.5% vs. 1.5%). Discontinuation due to adverse events regardless of causality occurred in 23 (17.8%) patients in the surufatinib group and 4 (5.9%) patients in the placebo group, including urine protein present (2.3% vs. 0%), proteinuria (1.6% vs. 0%) and the rest were in one patient (0.8%) each.

Rates of on-treatment deaths (defined as from the first dose until 37 days after the last dose) were similar between both groups: 3 (2.3%) in the surufatinib group and 2 (2.9%) in the placebo group. One death occurring in the placebo group was attributed to disease progression; the remaining deaths (3 [(2.3%] for surufatinib and 1 [1.5%] for placebo) were attributed to adverse events. Two adverse events leading to death (disseminated intravascular coagulation and liver injury, respectively) in the surufatinib group were assessed possibly related to the study drug by investigators.

### DISCUSSION

This phase III trial was the first study of advanced NETs to include tumors originating from any extra-pancreatic location. That is, patients were included with uncommon NETs when designing this trial, as studies of uncommon extra-pancreatic NETs, namely those originating from outside the gastrointestinal tract, lung, and thymus, were lacking due to low incidence and represented an unmet medical need. In addition, identifying the precise origin of NETs can prove challenging due to the heterogeneous nature of disease and limitations in the pathological and imaging techniques currently available. In this study, the primary tumor origin was unknown for 14% of patients, which was comparable to other previous studies. A post-hoc subgroup analysis of patients with NETs of unknown or uncommon tumor origins, indicated a clear benefit for those patients randomized to surufatinib, with a HR for median progression-free survival of 0.50 (95% CI 0.24, 1.06).

In contrast to previous studies, such as Alliance A021202 (pazopanib) and RADIANT-4 (everolimus), this phase III trial was conducted in Chinese patients with NETs of majority grade 2. This cohort is consistent with the clinicopathologic characteristics of real-world population of Chinese NETs patients in the metastatic setting, as reported in epidemiological studies. Although most NETs in this study were pathologically grade 2, and a higher percentage of tumors originated from the rectum rather than from the small intestine, which was associated with poorer prognosis, the progression-free survival favored surufatinib over placebo across all subgroups.

Prolonged progression-free survival with surufatinib was also demonstrated in patients previously treated with chemotherapy or SSAs. Despite a small sample size (7.8% and 11.6% of patients in the surufatinib and placebo groups, respectively), patients previously treated with everolimus achieved a median progression-free survival of 7.4 months with surufatinib versus 2.1 months with placebo.

Image evaluation of NETs remains challenging for both diagnosis and therapeutic monitoring. The characteristics of metastatic hepatic lesions of NETs in CT/MRI may confound assessment of disease progression (e.g., equidensity at baseline, low-density after treatment). In addition, the response evaluation of NETs usually requires the engagement of multiple structural imaging technologies, plus molecular imaging techniques. In this study, many patients had received prior loco-regional therapy. A lack of access to the loco-regional treatment information or prior images posed difficulties for central image readers. Despite these challenges, a similar trend of superior surufatinib efficacy was observed between investigator- and BIIRC-assessed results. Furthermore, analysis of post-hoc, independent, blinded image adjudication, performed to resolve the discrepancy between investigator and BIIRC assessments, consistently indicated that surufatinib treatment significantly prolonged progression-free survival (HR 0.57, 95% CI 0.38, 0.85).

The safety profile of surufatinib in this study was consistent with other previous studies. Most treatment-related adverse events were mild-to-moderate. The most common treatment-related grade 3 or higher adverse events were hypertension and proteinuria, which are known to be associated with this class of agent. The low incidence of skin reactions, such as hand-foot syndrome, may differentiate surufatinib from other agents in the class. Patient-reported outcomes demonstrated that quality of life for surufatinib-treated patients was comparable to those treated with placebo.

As compared with other trials involving patients with extra-pancreatic NETs, the population enrolled in this study was heterogenous. In addition, only Chinese patients were enrolled in this study, which may limit the extrapolation of the results to other populations: however, historically no ethnic differences in the efficacy of antiangiogenic agents for NETs have been reported. Furthermore, encouraging results were observed in a phase I study of surufatinib treating patients in the US, with objective response rate of 13.3% ( 2/15) and disease control rate of 73.3% ( 11/15) in advanced pancreatic NETs.

Surufatinib significantly prolonged progression-free survival, produced superior objective response and disease control rates compared to placebo, and was well-tolerated in most patients with progressive, advanced extra-pancreatic NETs.

What is claimed:

1. A method of treating advanced well-differentiated pancreatic neuroendocrine tumors in a patient in need thereof, the method comprising:
administering to the patient an effective amount of surufatinib or a pharmaceutically acceptable salt thereof.

2. The method of claim 1, wherein the patient was previously treated with no more than two prior first-line or second-line antitumor treatment chosen from somatostatin analogues (SSA), interferon, peptide receptor radionuclide therapy (PRRT), mammalian rapa Mycomycin target protein (mTOR) inhibitors and chemotherapy; and after the first-line or second-line antitumor treatment, the patient's neuroendocrine tumors continued to progress, wherein the patient with functioning NETs requiring treatment with long-acting SSAs, progression on prior VEGF/VEGFR inhibitors, or unstable or uncontrolled brain metastases were excluded.

3. The method of claim 1, wherein the patient does not have moderate or severe liver dysfunction.

4. The method of claim 1, wherein the patient does not have moderate or severe renal insufficiency.

5. The method of claim 1, wherein the effective amount of surufatinib is a dose of 300 mg, administered orally to the patient once daily (QD).

6. The method of claim 1, wherein the effective amount of surufatinib is a dose of less than 300 mg, administered orally to the patient once daily (QD).

7. The method of claim 6, wherein the effective amount of surufatinib ranges from 200 mg to 300 mg.

8. The method of claim 7, wherein the effective amount of surufatinib may be chosen from 300 mg, 250 mg, 200 mg, or a combination thereof.

9. The method of claim 1, wherein the effective amount of surufatinib is a dose taken as a single administration per day.

10. The method of claim 1, wherein the administering step occurs at the same time every day.

11. The method of claim 1, wherein administrating surufatinib occurs continuously.

12. The method of claim 11, wherein administering surufatinib is continuous over a treatment cycle and the treatment cycle continues until the neuroendocrine tumor progression stops or the patient suffers from intolerable toxicity of surufatinib.

13. The method of claim 12, wherein the treatment cycle is up to about 4 weeks.

14. The method of claim 13, wherein the treatment cycle is from one to four weeks.

15. The method of claim 1, wherein the effective amount of surufatinib is a dose administered once daily and further comprises adjusting the dose according to the safety and tolerability of the patient.

16. The method of claim 15, wherein adjusting the dose is chosen from dose interruption, dose reduction, dose discontinuation, and no dose adjustment.

17. The method of claim 16, wherein adjusting the dose is dose interruption and dose interruption occurs when the patient meets one or more criteria chosen from:
Grade 2 bleeding from any part,
24-hour urine protein quantity 22.0 g,
Grade 2 acute renal injury,
increased Grade 2 transaminase in combination with increased Grade 1 bilirubin, and
adverse reactions of Grade 3 or Grade 4 except those requiring permanent discontinuation.

18. The method of claim 16, wherein adjusting the dose is dose reduction and dose reduction occurs
when an adverse reaction resolves to ≤Grade 1 within 4 weeks, a first dose is adjusted to 250 mg of surufatinib QD and a second dose is adjusted to 200 mg of surufatinib QD; and
when a dose of 200 m of surufatinib QD is still intolerable, a dose adjustment to 200 mg surufatinib QD for 3 weeks on and 1 week off.

19. The method of claim 16, wherein adjusting the dose is dose discontinuation and dose discontinuation occurs when the patient meets one or more criteria chosen from:
hemorrhage or gastrointestinal perforation ≥Grade 3;
nephrotic syndrome or hypertension crisis;
transaminase ≥3×ULN in combination with bilirubin increased to ≥2×ULN;
increased Grade 4 transaminase in combination with increased Grade 4 bilirubin; and
arterial thrombosis.

20. A method of treating pancreatic neuroendocrine tumors or advanced well-differentiated pancreatic neuroendocrine tumors in a patient in need thereof, the method comprising:
administering to the patient a pharmaceutical composition comprising surufatinib or a pharmaceutically acceptable salt thereof and at least one additional component chosen from pharmaceutically acceptable carriers, pharmaceutically acceptable vehicles, and pharmaceutically acceptable excipients.

21. A method of treating advanced well-differentiated extra-pancreatic neuroendocrine tumors in a patient in need thereof, the method comprising:
administering to the patient an effective amount of surufatinib or a pharmaceutically acceptable salt thereof.

22. The method of claim 21, wherein the patient was previously treated with one or more first-line or second-line antitumor treatment chosen from one or more of somatostatin analogues (SSA), interferon, peptide receptor radionuclide therapy (PRRT), mammalian rapa Mycomycin target protein (mTOR) inhibitors and chemotherapy; and after the first-line or second-line antitumor treatment, the patient's neuroendocrine tumors continued to progress.

23. The method of claim 21, wherein the patient does not have moderate or severe liver dysfunction.

24. The method of claim 21, wherein the patient does not have moderate or severe renal insufficiency.

25. The method of claim 21, wherein the effective amount of surufatinib is a dose of 300 mg, administered orally to the patient once daily (QD).

26. The method of claim 21, wherein the effective amount of surufatinib is a dose of less than 300 mg, administered orally to the patient once daily (QD).

27. The method of claim 26, wherein the effective amount of surufatinib ranges from 200 mg to 300 mg.

28. The method of claim 27, wherein the effective amount of surufatinib may be chosen from 300 mg, 250 mg, 200 mg, or a combination thereof.

29. The method of claim 21, wherein the effective amount of surufatinib is a dose taken as a single administration per day.

30. The method of claim 21, wherein the administering step occurs at the same time every day.

31. The method of claim 21, wherein administrating surufatinib occurs continuously.

32. The method of claim 31, wherein administering surufatinib is continuous over a treatment cycle and the treatment cycle continues until the neuroendocrine tumor progression stops or the patient suffers from intolerable toxicity of surufatinib.

33. The method of claim 32, wherein the treatment cycle is up to about 4 weeks.

34. The method of claim 33, wherein the treatment cycle is from one to four weeks.

35. The method of claim 21, wherein the effective amount of surufatinib is a dose administered QD and further comprises adjusting the dose according to the safety and tolerability of the patient.

36. The method of claim 35, wherein adjusting the dose is chosen from dose interruption, dose reduction, dose discontinuation, and no dose adjustment.

37. The method of claim 36, wherein adjusting the dose is dose interruption and dose interruption occurs when the patient meets one or more criteria chosen from:
Grade 2 bleeding from any part,
24-hour urine protein quantity 22.0 g,
Grade 2 acute renal injury,
increased Grade 2 transaminase in combination with increased Grade 1 bilirubin, and
any adverse reactions of Grade 3 or Grade 4 except those requiring permanent discontinuation.

38. The method of claim 37, wherein administration is reinitiated when one or more of the criteria resolves to ≤Grade 1 within one week after the dose interruption.

39. The method of claim 36, wherein adjusting the dose is dose reduction and dose reduction occurs
when an adverse reaction resolves to ≤Grade 1 within 4 weeks, a first dose is adjusted to 250 mg of surufatinib QD and a second dose is adjusted to 200 mg of surufatinib QD; and
when a dose of 200 m of surufatinib QD is still intolerable, a dose adjustment to 200 mg surufatinib QD for 3 weeks on and 1 week off.

40. The method of claim 36, wherein adjusting the dose is dose discontinuation and dose discontinuation occurs when the patient meets one or more criteria chosen from:
hemorrhage or gastrointestinal perforation ≥Grade 3;
nephrotic syndrome or hypertension crisis;
transaminase ≥3×ULN in combination with bilirubin increased to ≥2×ULN;
increased Grade 4 transaminase in combination with increased Grade 4 bilirubin; and
arterial thrombosis.

41. The method of claim 21, further comprising adjusting the effective amount of surufatinib administered per day according to a proteinuria level in the patient.

42. The method of claim 41, wherein adjusting the dose is no dose adjustment and no dose adjustment occurs when the patient meets one or more criteria chosen from: when urinalysis shows protein + and 24-hour urine protein quantity is less than 1.0 g, and when urinalysis shows protein 2+ or 3+ and 24-hour urine protein quantity is 1.0-2.0 g, excluding 2.0 g.

43. The method of claim 41, wherein adjusting the effective amount of surufatinib occurs when the patient meets one or more criteria chosen from:
when a first 24-hour urine protein quantity ≥22.0 g occurs, a dose interruption applies, and the dose of surufatinib is reduced to 250 mg if the test results resolve to ≤Grade 1 within 4 weeks;
when a second 24-hour urine protein quantity ≥22.0 g occurs, the dose interruption applies, and the dose of surufatinib is reduced to 200 mg if the test results resolve to ≤Grade 1 within 4 weeks; and
when a third 24-hour urine protein quantity ≥22.0 g occurs, the dose interruption applies, and the dose of surufatinib is reduced to 200 mg with 3 weeks on and 1 week off if the test results resolve to ≤Grade 1 within 4 weeks, or dose discontinuation applies.

44. A method of treating extra-pancreatic neuroendocrine tumors or advanced well-differentiated extra-pancreatic neuroendocrine tumors in a patient in need thereof, the method comprising:
administering to the patient a pharmaceutical composition comprising surufatinib or a pharmaceutically acceptable salt thereof and at least one additional component chosen from pharmaceutically acceptable carriers, pharmaceutically acceptable vehicles, and pharmaceutically acceptable excipients.
